SEQUENCE LISTING (17 sequences)

SEQ ID NO: 1 - DNA, 30 bp, Artificial Sequence
  Single stranded oligonucleotide to detect EGFR deletion
tcgctgctttcggagatgttttgatagcga

SEQ ID NO: 2 - DNA, 31 bp, Artificial Sequence
  Single stranded oligonucleotide to detect EGFR deletion
tcgctgctttcggagaatgtcttgatagcga

SEQ ID NO: 3 - DNA, 28 bp, Artificial Sequence
  Single stranded oligonucleotide to detect EGFR deletion
tcgctggctttcgattccttgatagcga

SEQ ID NO: 4 - DNA, 23 bp, Artificial Sequence
  Single stranded oligonucleotide to detect EGFR mutation
cagattggcccgcccaaaatctg

SEQ ID NO: 5 - DNA, 29 bp, Artificial Sequence
  Single stranded oligonucleotide to detect EGFR mutation
tgcaggcatgagctgcatgatgagctgca

SEQ ID NO: 6 - DNA, 25 bp, Artificial Sequence
  Single stranded oligonucleotide to use as control
cacgtcgacaagcgaccgatacgtg

SEQ ID NO: 7 - DNA, 23 bp, Artificial Sequence
  Single stranded oligonucleotide to detect survivin expression
tggtccttgagaaagggcgacca

SEQ ID NO: 8 - DNA, 24 bp, Artificial Sequence
  Single stranded oligonucleotide to detect H5 flu virus and its infection
ctgagtcccctttcttgacctcag

SEQ ID NO: 9 - DNA, 24 bp, Artificial Sequence
  Single stranded oligonucleotide to detect N1 flu virus and its infection
cacacatgcacattcagacgtgtg

SEQ ID NO: 10 - DNA, 24 bp, Artificial Sequence
  Single stranded oligonucleotide to detect FluA virus and its infection
cgtgctgctgtttggaattgcacg

SEQ ID NO: 11 - DNA, 25 bp, Artificial Sequence
  Single stranded oligonucleotide to detect FluB and its infection
cgttctgtcgtgcattataggaacg

SEQ ID NO: 12 - DNA, 30 bp, Artificial Sequence
  Probe type specific to fluAH5 conserved sequence
gcatacaaaattgtcaagaaaggggactca

SEQ ID NO: 13 - DNA, 30 bp, Artificial Sequence
  Probe type specific to fluAN1 conserved sequence
agaactcaagagtctgaatgtgcatgtgta

SEQ ID NO: 14 - DNA, 30 bp, Artificial Sequence
  Probe type specific to fluA conserved sequence
ctcaaagggaaattccaaacagcagcacaa

SEQ ID NO: 15 - DNA, 30 bp, Artificial Sequence
  Probe type specific to fluB conserved sequence
tgctttcctataatgcacgacagaacaaaa

SEQ ID NO: 16 - DNA, 5370 bp, Artificial Sequence
  EGFR sequence for corresponding mutation and deletion positions where ALV-EGFR molecules detect.
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 17 - DNA, 24 bp, Artificial Sequence
  Single stranded oligonucleotide to detect survivin expression
ctgagaaagggctgccagtctcag

(19) United States
(12) Patent Application Publication
(10) Pub. No.: US 2008/0032282 A1
(43) Pub. Date: Feb. 7, 2008

(54) Methods and applications of molecular beacon imaging for infectious disease and cancer detection

(75) Inventors: Augustine Lin, San Ramon, CA (US); Pan-Chyr Yang, Taipei City (TW); Cheng-Chung Chou, Xindian City (TW)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: AL Vitae Pharmaceuticals, San Ramon, CA (US)

(21) Appl. No.: 11/644,400

(22) Filed: Dec. 22, 2006

Related U.S. Application Data
(60) Provisional application No. 60/753,651, filed on Dec. 23, 2005.
(60) Provisional application No. 60/753,960, filed on Dec. 23, 2005.

(51) Int. Cl. C12Q 1/70; C12Q 1/68

(52) U.S. Cl. 435/5; 435/6

(57) ABSTRACT

Molecular beacon for detecting an infection and/or expression or a mutation of a disease marker for diagnostics and pharmacogenomics. The molecular beacon is capable of hybridizing a disease-related RNA or DNA of a disease marker in a specimen obtained from a living subject, thereby emitting a signal detectable without a need for signal amplification. The disease marker includes a genetic sequence specific to a pathogen including a flu virus, a cancer cell marker, and a drug resistance-related genetic mutation marker for a drug resistant cancer and infectious pathogen. To detect a disease cell, a specimen containing one or more cells is obtained from a living subject, and fixed by an organic solvent. A molecular beacon is then added to the specimen, followed by staining nuclei of the cells in the specimen. The signal is detectable with a microscope, FACS scan, ELISA plate reader, Scanner, or any combinations thereof.

### CROSS-REFERENCE TO RELATED PATENT APPLICATION

This application claims the benefit, pursuant to 35 U.S.C. §119(e), of U.S. provisional patent application Nos. 60/753,651 filed Dec. 23, 2005, entitled “METHODS AND APPLICATIONS OF MOLECULAR BEACON IMAGING FOR INFECTIOUS DISEASE AND CANCER DETECTION” by Augustine Lin, Pan-Chyr Yang, and Cheng-Chung Chou, and 60/753,960, filed Dec. 23, 2005, entitled “METHODS AND APPLICATIONS OF MOLECULAR BEACON IMAGING FOR IDENTIFYING AND VALIDATING GENOMIC TARGETS, AND FOR DRUG SCREENING,” by Augustine Lin, which are incorporated herein by reference in their entireties.

This application is related to a co-pending U.S. patent application, entitled “METHODS AND APPLICATIONS OF MOLECULAR BEACON IMAGING FOR IDENTIFYING AND VALIDATING GENOMIC TARGETS, AND FOR DRUG SCREENING,” by Augustine Lin, (Attorney Docket No. 16957-58761). The above identified co-pending application was filed on the same day that this application was filed, and with the same assignee as that of this application. The disclosure of the above identified co-pending application is incorporated herein by reference in its entirety.

Some references, which may include patents, patent applications and various publications, are cited and discussed in the description of this invention. The citation and/or discussion of such references is provided merely to clarify the description of the present invention and is not an admission that any such reference is “prior art” to the invention described herein.

All references cited and discussed in this specification are incorporated herein by reference in their entireties and to the same extent as if each reference was individually incorporated by reference. In terms of notation, hereinafter, “[n]” represents the nth reference cited in the reference list. For example, [3] represents the 3rd reference cited in the reference list, namely, Giesendorf B A J et al. Molecular beacons: a new approach for semi-automated mutation analysis. Clin Chem 1998; 44:482-486.

### FIELD OF THE INVENTION

The present invention relates generally to molecular beacons for detection of a disease marker, and more specifically to molecular beacons for detection of an infection and/or expression or a mutation of a disease marker and methods of using the same for diagnosis and pharmacogenomics in a living subject.

### BACKGROUND OF THE INVENTION

Cancer is the second leading cause of death in the United States. Nearly half of all men and a little over one third of all women in the United States will develop cancer during their lifetimes. Today, millions of people are living with cancer or have had cancer. A crucial factor to increase patients' survival is to diagnose cancer early. The sooner a cancer is found and treatment begins, the better are the chances for living for many years. At present, there is no reliable serum tumor marker for diagnosis of cancer. As an example, in the case of breast cancer, although early screening with mammography decreased the mortality of the disease, nearly 20% of breast cancer patients are still missed by mammography. Furthermore, of all patients with abnormal mammograms, only 10 to 20% were confirmed to have breast cancer by biopsy. Therefore, development of novel approaches to early diagnosis of cancer is of critical importance for the successful treatment and for increasing survival of the patients. Development of new approaches to the early detection of cancer cells and the determination of the responses of the cancer cells to therapeutic reagents holds great promise to increase the survival of cancer patients.

Like cancer being a threat to the human, infectious disease is also a leading cause of death, accounting for a quarter to a third of deaths worldwide. New and reemerging infectious diseases will pose a rising global health threat and will complicate global security over the next 20 years. The current outbreak of highly pathogenic avian flu, which began in Southeast Asia in mid-2003 are the largest and most severe on record. Never before in the history of this disease have so many countries been simultaneously affected, resulting in the loss of so many birds. The causative agent, H5N1 virus, has proved to be especially tenacious. Experts at WHO and elsewhere believe that the world is now closer to another influenza pandemic than at any time since 1968, when the last of the previous century's three pandemics pandemics occurred. CDC has recommended strong measures to detect (domestic surveillance), diagnose, and laboratory testing for H5N1 to prevent the spread of avian fluA (H5N1) virus. Due to the widespread epidemic of avian H5N1 influenza in birds and possible bird-to-human transmission of avian H5N1 virus, an early and sensitive diagnostic method for detecting avian flu as well as human flu virus is in urgently demanding.

The lack of effective early pharmacogenomic detection has often attributed to the difficulty in the treatment for many life-threatening diseases. A rapid, accurate, specific and affordable diagnosis and/or pharmacogenomics screen in the early stage of a disease progression can provide invaluable benefits to patients with improvement in outcome and to physicians in decision making regarding the optimal treatment for patients.

Molecular beacons (MB) are hybridization probes that can be used to detect the presence of complementary nucleic acid targets without having to separate probe-target hybrids from excess probes in hybridization assays [15, 16]. Because of this property, MB have been used for the detection of RNAs within living cells [10, 13], for monitoring the synthesis of specific nucleic acids in sealed reaction vessels [6, 16,], and for the construction of self-reporting oligonucleotide arrays [14]. MB can be used to perform homogeneous one-tube assays for identification of single-nucleotide variations in DNA [3, 7-9] and for detection of pathogens [12, 17].

Although previous studies demonstrated that detection of the presence of complementary nucleic acid targets using MB probes is a feasible approach, the question remains, among other things, how to develop this novel technology into a simple procedure that can be used broadly in basic research and clinical laboratories.

Therefore, a heretofore-unaddressed need exists in the art to address the aforementioned deficiencies and inadequacies.

### SUMMARY OF THE INVENTION

The present invention, among other things, seeks to solve aforementioned deficits present in currently available methods of using a molecular beacon as a diagnostic agent for detecting a disease cell, in particular, for detection of an infectious disease cell and/or a cancer cell.

In one aspect, the present invention relates to a method for detecting an infection and/or expression or a mutation of a disease marker for diagnostics and pharmacogenomics in a living subject. In one embodiment, the method comprises the steps of a) obtaining a specimen from the living subject, wherein the specimen contains one or more cells; b) fixing specimen with an organic solvent; c) adding a molecular beacon to the specimen; and d) observing a result from adding the molecular beacon for detection of an infection and/or expression or a mutation of a disease marker, wherein the molecular beacon is capable of hybridizing with a disease-related RNA or DNA of the disease marker in the one or more cells, thereby emitting a signal detectable without a need for signal amplification. The method further comprises the step of staining at least one nuclei of one or more cells with a stain prior to the observing step.

Performing the steps of adding the molecular beacon and observing the result takes no more than 2 hours.

The staining result is detectable with an instrument including one of a microscope, FACS scan, ELISA plate reader, Scanner, and any combinations thereof.

The molecular beacon can detect an infectious disease cell, wherein the infectious disease comprises a flu virus disease. The flu virus includes a fluA virus, wherein the fluA virus includes one of 16H and 9N strains, and any combinations thereof. The flu virus can also be selected from the group consisting of fluA, fluAH5, fluAN1, fluB, and any combinations thereof.

The molecular beacon can also detect a cancer cell, wherein the cancer is selected from the group consisting of lung cancer, liver cancer, stomach cancer, prostate cancer, breast cancer, pancreatic cancer, skin cancer, bone cancer, womb cancer, cervical cancer, brain cancer, colon cancer, throat cancer and any cancer occurred in an animal.

The mutation is a point mutation and/or deletion of the disease marker, wherein the disease marker is a biological target of a targeted therapeutics. The biological target is EGFR gene and/or a transcription product thereof, wherein the EGFR gene contains a deletion mutation in EGFR tyrosine kinase domain.

In one embodiment, the molecular beacon comprises a single stranded hairpin shaped structured oligonucleotide probe containing a nucleotide sequence selected from the group consisting of SEQ ID NOs: 1-11, and any combinations thereof. In another embodiment, the molecular beacon is capable of hybridizing with a transcription product of EGFR. In yet another embodiment, the molecular beacon is capable of detecting a drug-resistant cancer and/or a drug-resistant pathogen.

In another aspect, the present invention relates to a method for detecting a cancer cell from a living subject. In one embodiment, the method comprises the steps of:
a) obtaining from the living subject a specimen containing one or more cells;
b) fixing the specimen with an organic solvent;
c) adding a molecular beacon into the specimen; and
d) observing a result from adding the molecular beacon for detection of the cancer cell in the specimen,
   wherein the molecular beacon is capable of hybridizing with a cancer cell marker-related RNA or DNA in one or more cells in the specimen, thereby emitting a signal detectable without a need for signal amplification. The method further comprises the step of staining a nuclei of one or more cells in the specimen with a stain prior to the observing step.

The organic solvent is one of acetone, alcohol, methanol, formalin, paraformaldehyde, butanol, and any combinations thereof, wherein the organic solvent fixed specimen is subject to a Triton treatment prior to the addition of the molecular beacon.

The molecular beacon comprises a single stranded hairpin shaped structured oligonucleotide probe containing a nucleotide sequence selected from the group consisting of SEQ ID NOs: 1-7, and any combinations thereof.

The cancer cell is selected from the group consisting of lung cancer, liver cancer, stomach cancer, prostate cancer, breast cancer, pancreatic cancer, skin cancer, bone cancer, womb cancer, cervical cancer, brain cancer, colon cancer, throat cancer and any cancer occurred in an animal, wherein the cancer cell exhibits at least one point mutation and/or deletion in a specific marker of the cancer cell.

The specimen is one of a tissue section, an aspirate from biopsy, blood, and an exfoliated cell in a body fluid.

In another aspect, the present invention relates to a method for detecting a flu virus-infected cell from a living subject. In one embodiment, the method comprises the steps of: obtaining from the living subject a specimen, wherein the specimen contains one or more cells; fixing specimen with an organic solvent; adding a molecular beacon into the specimen; observing the result for detection of the flu virus-infected cell in the specimen, wherein the molecular beacon is capable of hybridizing with a flu virus marker-related RNA or DNA in at least one cell, thereby emitting a signal detectable without a need for signal amplification. The method further comprises the step of staining at least one nuclei of one or more cells with a stain prior to the observing step.

In one embodiment, the organic solvent is one of acetone, alcohol, methanol, formalin, paraformaldehyde, butanol, and any combinations thereof, wherein the organic solvent fixed specimen is subject to a Triton treatment prior to addition of the molecular beacon.

In one embodiment, the molecular beacon comprises a single stranded hairpin shaped structured oligonucleotide probe containing a nucleotide sequence selected from the group consisting of SEQ ID NOs: 8-11, and any combinations thereof.

In one embodiment, the flu virus comprises an avian flu virus, wherein the flu virus is a fluA or fluB virus. The fluA virus includes one of 16H and 9N strains, and any combinations thereof.

In one embodiment, the method is practiced with one or more than one probe that are added into the specimen simultaneously.

In yet another aspect, the present invention relates to a method for detecting an infection and/or expression or a mutation of a disease marker for diagnostics and pharmacogenomics in a living subject. In one embodiment, the method comprises the steps of:

obtaining a specimen from the living subject, wherein the specimen contains one or more cells;

fixing specimen with an organic solvent;

adding a molecular beacon to the specimen;

observing a result from adding the molecular beacon for detection of infections and/or expression or a mutation of a disease marker,

wherein the molecular beacon is capable of hybridizing with a disease-related RNA or DNA of the disease marker in a cell, thereby emitting a signal detectable without a need for signal amplification, and wherein performing adding the molecular beacon and observing the result take no more than 2 hours.

In one embodiment, the method further comprises the step of staining a nuclei of one or more cells in the specimen with a stain prior to the observing step.

In one embodiment, the organic solvent is one of acetone, alcohol, methanol, formalin, paraformaldehyde, butanol, and any combinations thereof.

In a further aspect, the present invention relates to a molecular beacon comprising a single stranded hairpin shaped structured oligonucleotide probe containing a nucleotide sequence capable of hybridizing with a disease-related RNA and/or DNA of a disease marker in a disease cell, thereby emitting a signal detectable without a need for signal amplification.

In one embodiment, the oligonucleotide probe contains a nucleotide sequence selected from the group consisting of SEQ ID NOs: 1-11.

In yet another embodiment, the oligonucleotide probe has a nucleotide sequence capable of hybridizing with RNA and/or DNA encoding EGFR gene tyrosine kinase domain in a cancer cell.

In one embodiment, the oligonucleotide probe comprises a fluorofore at 5′ and a quencher at 3′, or a fluorofore at 3′ and a quencher at 5′.

In one embodiment, the disease cell is one of a cancer cell or an infectious disease cell.

In one embodiment, the disease cell is infected by a flu virus, wherein the flu virus is a fluA or fluB virus. In one embodiment, the fluA virus includes one of 16H and 9N strains, and any combinations thereof.

In yet another aspect, the present invention relates to a diagnostic kit for detecting an infection and/or expression or a mutation of a disease marker for diagnostics and pharmacogenomics in a living subject comprising:

a molecular beacon comprising a single stranded hairpin shaped structured oligonucleotide probe containing a nucleotide sequence capable of hybridizing with a disease-related RNA and/or DNA of a disease marker in a disease cell, thereby emitting a signal detectable without a need for signal amplification; and

an instruction sheet.

In one embodiment, the oligonucleotide probe contains a nucleotide sequence capable of hybridizing with RNA and/or DNA encoding EGFR gene tyrosine kinase domain in a cancer cell.

In one embodiment, the oligonucleotide probe contains a nucleotide sequence selected from the group consisting of SEQ ID NOs: 1-11.

In one embodiment, the kit comprises more than one oligonucleotide probe containing a nucleotide sequence selected from the group consisting of SEQ ID NOs: 1-7.

In one embodiment, the kit comprises more than oligonucleotide probe containing a nucleotide sequence selected from the group consisting of SEQ ID NOs: 8-11.

The diagnostic kit allows the performance of diagnosis from adding the molecular beacon into a specimen to observing a result therefrom to take no more than 2 hours. The methods provided by the invention afford advantages of diagnosis of a infection disease cell and/or a cancer cell in a rapid one-step assay with a high level of sensitivity, and/or being able to simultaneously detect mutations as well as expression of a specific therapeutic target or marker from a biological specimen.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

FIG. 1 shows the florescence of molecules designed for detection of cancer markers and targets of cancer pharmacogenomics according to one embodiment of the present invention.

FIG. 2 shows images of point mutations of a therapeutic target in lung cancer cell lines I and II detected with ALV-1011 according to one embodiment of the present invention.

FIG. 3 shows images of the second point mutations of a therapeutic target in lung cancer cell lines I and II detected with ALV-1022 according to one embodiment of the present invention.

FIG. 4 shows expressions of a “universal” cancer marker in lung cancer cell lines I and II detected with ALV-1033 according to one embodiment of the present invention.

FIG. 5 shows images of point mutations of a cancer marker in biopsies of pancreatic cancer patient detected with ALV-1044 and ALV-1055 according to one embodiment of the present invention.

FIG. 6 shows specific binding of ALV-FluA, ALV-FluAH5, ALV-FluAN1 and ALV-FluB molecules to their respective targets according to one embodiment of the present invention.

FIG. 7 shows fluA, fluAH5 and fluAN1 detected in avian flu virus infections according to one embodiment of the present invention.

FIG. 8 shows fluA and fluB detected in human flu virus infections according to one embodiment of the present invention.

FIG. 9 shows human fluA and fluB infection rapidly detected in 10-20 minutes according to one embodiment of the present invention.

FIG. 10 shows FACS analysis of human fluA and fluB virus infection detected by ALV-FluA and ALV-FluB molecules according to one embodiment of the present invention.

FIG. 11 shows fluorescent microscope analysis of human fluA and fluB virus infection detected by ALV-FluA and ALV-FluB molecules according to one embodiment of the present invention.

FIG. 12 shows target binding of ALV-FluA, ALV-FluB, ALV-FluAH5 and ALV-FluAN1 molecules.

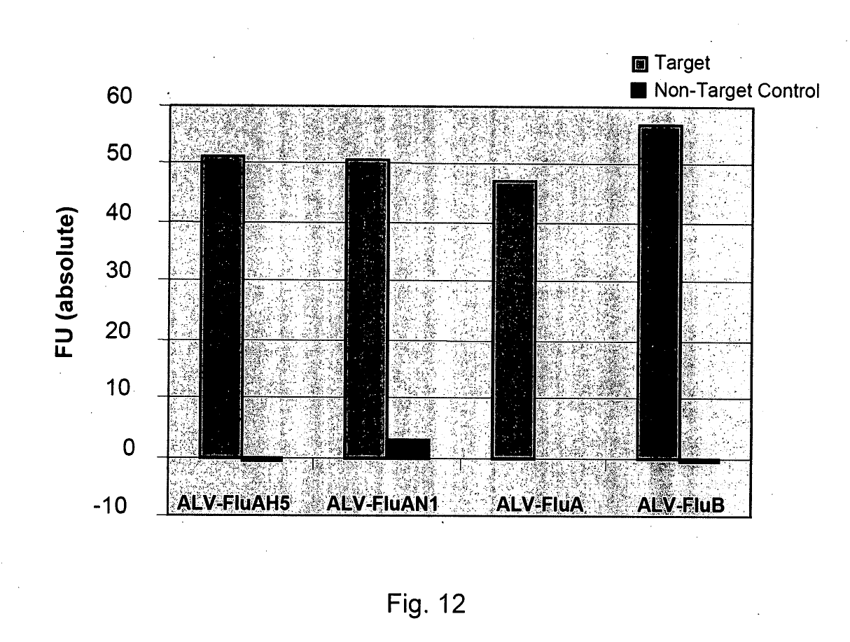

FIG. 13 shows ALV-FluA detection of human fluA virus infection according to one embodiment of the present invention.

FIG. 14 shows ALV-FluB detection of human fluB virus infection according to one embodiment of the present invention.

FIG. 15 shows ALV-FluH5 detection of human fluH5 virus infection according to one embodiment of the present invention.

FIG. 16 shows ALV-FluAN1 detection of Avian fluAN1 virus infection according to one embodiment of the present invention.

FIG. 17 shows ALV-FluA detection of avian fluA virus infection according to one embodiment of the present invention.

FIG. 18 shows FACS analysis of flu virus infection following ALV-FluA detection according to one embodiment of the present invention.

FIG. 19 shows RFU analysis of human flu virus infection with fluorescence plate reader according to one embodiment of the present invention.

FIG. 20 shows detection of flu virus infection in cell cultures according to one embodiment of the present invention.

FIG. 21 shows detection of flu virus infection in a patient according to one embodiment of the present invention.

FIG. 22 shows detection of avian flu fluA(H5N3) infection in chicken embryonic cells according to one embodiment of the present invention.

FIG. 23 shows detection of avian flu fluA(H6N1) infection in chicken embryonic cells according to one embodiment of the present invention.

FIG. 24 shows detection of point mutations of a therapeutic target in lung cancer cell line I according to one embodiment of the present invention.

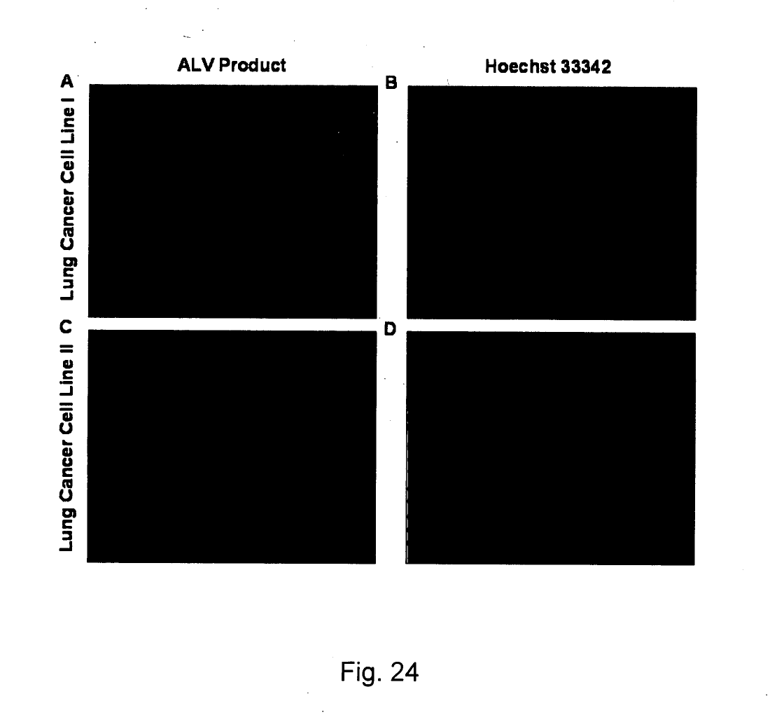

FIG. 25 shows detection of deletions of a therapeutic target in lung cancer cell line III according to one embodiment of the present invention.

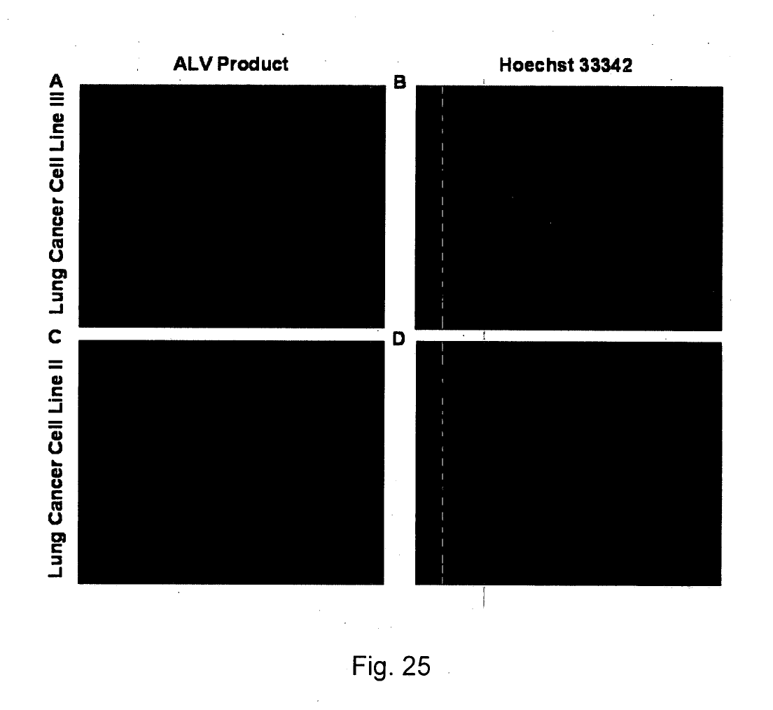

FIG. 26 shows detection of mutations in SMCLC patients according to one embodiment of the present invention.

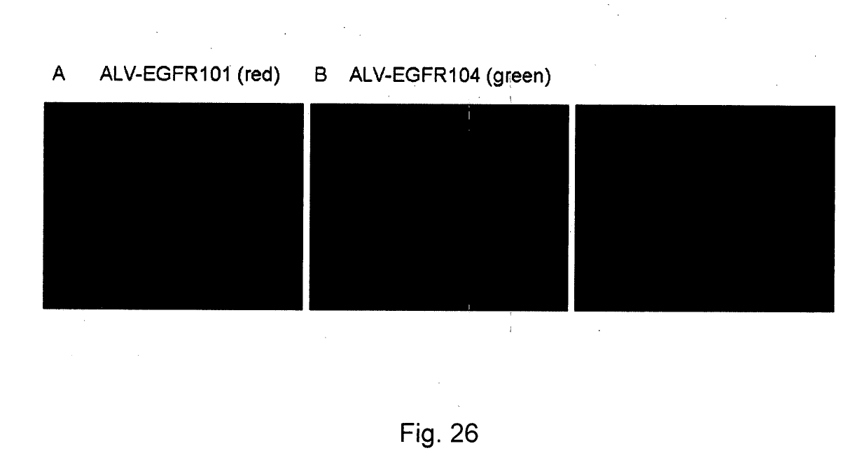

FIG. 27 shows the nucleotide sequence that is specific to flu virus types of fluA and fluB, and strains of fluAH5 and fluAN1 according to one embodiment of the present invention, which shows positions of EGFR point mutations and deletions where ALV EGFR MBs detect for cancer pharmacogenomics.

FIG. 28 shows sequences identified by bioinformatics that are specific to flu virus types of fluA and fluB, and strains of fluAH5 and fluAN1.

### DETAILED DESCRIPTION OF THE PREFERRED EMBODIMENTS

The present invention is more particularly described in the following examples that are intended as illustrative only since numerous modifications and variations therein will be apparent to those skilled in the art. Various embodiments of the invention are now described in detail. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention.

### DEFINITIONS

The terms used in this specification generally have their ordinary meanings in the art, within the context of the invention, and in the specific context where each term is used. Certain terms that are used to describe the invention are discussed below, or elsewhere in the specification, to provide additional guidance to the practitioner in describing the compositions and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification.

As used herein, “about” or “approximately” shall generally mean within 20 percent, preferably within 10 percent, and more preferably within 5 percent of a given value or range. Numerical quantities given herein are approximate, meaning that the term “about” or “approximately” can be inferred if not expressly stated.

“Hybridization” and “complementary” as used herein, refer to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary or hybridizable to each other at that position. The oligonucleotide and the DNA or RNA hybridize when a sufficient number of corresponding positions in each molecule are occupied by nucleotides which can hydrogen bond with each other. It is understood in the art that the sequence of an antisense oligonucleotide need not be 100% complementary to that of its target nucleic acid to hybridize thereto. An oligonucleotide is specifically hybridizable when binding of the compound to the target DNA or RNA molecule, and there is a sufficient degree of complementarity to avoid non-specific binding of the antisense oligonucleotide to non-target sequences under conditions in which specific binding is desired, e.g., under physiological conditions in the case of in vivo assays or therapeutic treatment, or, in the case of in vitro assays, under conditions in which the assays are performed.

In the context of this invention, the term “oligonucleotide” refers to an oligomer or polymer of ribonucleic acid (RNA) or deoxyribonucleic acid (DNA) or mimetics thereof. This term includes, but is not limited to, oligonucleotides composed of naturally occurring and/or synthetic nucleobases, sugars, and covalent internucleoside (backbone) linkages. Such modified or substituted oligonucleotides are often preferred over native forms because of desirable properties such as, for example, enhanced cellular uptake, enhanced affinity for nucleic acid targets, and/or increased stability in the presence of nucleases.

The term, as used herein, “molecular beacons” or its acronym “MBs” are single-stranded oligonucleotide hybridization probes that form a stem-and-loop structure. The loop contains a probe sequence that is complementary to a target sequence, and the stem is formed by the annealing of complementary arm sequences that are located on either side of the probe sequence. A fluorophore is covalently linked to the end of one arm and a quencher is covalently linked to the end of the other arm. Molecular beacons do not fluoresce when they are free in solution. However, when they hybridize to a nucleic acid strand containing a target sequence they undergo a conformational change that enables them to fluoresce brightly. In the absence of targets, the probe is dark, because the stem places the fluorophore so close to the nonfluorescent quencher that they transiently share electrons, eliminating the ability of the fluorophore to fluoresce. When the probe encounters a target molecule, it forms a probe-target hybrid that is longer and more stable than the stem hybrid. The rigidity and length of the probe-target hybrid precludes the simultaneous existence of the stem hybrid. Consequently, the molecular beacon undergoes a spontaneous conformational reorganization that forces the stem hybrid to dissociate and the fluorophore and the quencher to move away from each other, restoring fluorescence.

When the MB encounters a target mRNA molecule, the loop and a part of the stem hybridize to the target mRNA, causing a spontaneous conformational change that forces the stem apart. The quencher moves away from the fluorophore, leading to the restoration of fluorescence. One major advantage of the stem-loop probes is that they can recognize their targets with a higher specificity than the linear oligonucleotide probes. Properly designed MBs can discriminate between targets that differ by as little as a single nucleotide. The MBs have been utilized in a variety of applications including DNA mutation detection, protein-DNA interactions, real-time monitoring of PCR, gene typing and mRNA detection in living cells.

The terms “transfection” as used herein refers to the process of inserting a nucleic acid into a host. Many techniques are well known to those skilled in the art to facilitate transfection of a nucleic acid into a prokaryotic or eukaryotic organism. These methods involve a variety of techniques, such as treating the cells with high concentrations of salt such as, but not only calcium or magnesium salt, an electric field, detergent, or liposome mediated transfection, to render the host cell competent for the uptake of the nucleic acid molecules.

The term “gene” or “genes” as used herein refers to nucleic acid sequences (including both RNA and DNA) that encode genetic information for the synthesis of a whole RNA, a whole protein, or any portion of such whole RNA or whole protein.

The term “expressed” or “expression” as used herein refers to the transcription from a gene to give an RNA nucleic acid molecule at least complementary in part to a region of one of the two nucleic acid strands of the gene. The term “expressed” or “expression” as used herein may also refer to the translation from said RNA nucleic acid molecule to give a protein or polypeptide or a portion thereof.

As used herein, the term “pharmacogenomics” refers to a science that examines the inherited variations in genes that dictate drug response and explores the ways these variations can be used to predict whether a patient will have a good response to a drug, a bad response to a drug, or no response at all.

USMD™, an abbreviation of “Ultra Sensitive Molecular Detection,” is the trade name of the platform technology of the present invention.

### OVERVIEW OF THE INVENTION

One aspect of the present inventions relates to using a MB to detect an infection and expression or a mutation of a disease marker for diagnostics and pharmacogenomics by directly adding a MB to a specimen and obtain a signal detectable without a need for signal amplification. No product on the market can work so fast with the level of sensitivity and specificity achieved by the present invention. In the art, molecular beacons have been used with signal amplification. The molecular beacons and the methods provided by the invention can detect a signal without a need for signal amplification. Furthermore, the present invention takes no more than 2 hours to run a diagnosis, whereas the current standard molecular detection of flu recommended by WHO takes 6 hours, Moreover, the sequences of MBs of the present invention for cancer and flu have not been used, especially for flu sequences.

The present invention provides methods for detecting cancer and infectious diseases in sample cells. Specifically, provided herein are methods for detecting, identifying or quantitating the presence of, or alterations in a cancer marker sequence or in a virus marker sequence in a sample of cells.

One aspect of the invention relates to detecting an expressional change and/or a mutation of a disease specific marker directly from a tissue sample with no necessity of amplification. This platform technology provides advantages of sensitive, specific and simultaneous detection of multiple disease related markers. Delivering a MB containing reagent of the invention into a disease-associated cell will result in a change in signal. When the testing reagent detects the change in the molecular marker of a disease, e.g., expressional abnormalities or mutations, a disease cell (bright color) can be distinguished from a normal cell (dark color). Thus, integrating this breakthrough technology with the knowledge of functional genomics advanced in recent years, it is initially aimed to develop products for: 1) early detection of both acute and chronic diseases; 2) pharmacogenomic screening of patients to improve efficacy of therapeutic treatment; 3) prognosis and post treatment progression follow up of patients. The platform technology provides advantages of rapid, sensitive, specific and cost effective detection of disease related markers.

One aspect of the invention relates to a method for detecting an infection and/or expression or a mutation of a disease marker for diagnostics and pharmacogenomics in a living subject, in which the method includes: (a) obtaining a specimen from the living subject, in which the specimen contains one or more cells; (b) fixing specimen with an organic solvent; (d) adding a molecular beacon to the specimen; and (c) observing a result for detecting an infection and/or expression or a mutation of a disease marker.

The specimen containing cells of interest may be a tissue section, an aspirate from biopsy, blood, or an exfoliated cell in a body fluid. The specimen is fixed by an organic solvent before adding the MB. The organic solvent for fixing a specimen includes one of acetone, alcohol, methanol, formalin, paraformaldehyde, butanol, and any combinations thereof. In one embodiment, the organic solvent-fixed specimen is subject to a Triton treatment prior to the addition of a molecular beacon.

Optionally, before observing the result from adding the molecular beacon, the method may further include the step of staining at least one nuclei of one or more cells in the specimen with a stain. The staining of nuclei of the cells in the specimen makes it very easy to locate where the cells are on the slide.

The result from adding the molecular beacon is detectable with a suitable instrument including one of a microscope, FACS scan, ELISA plate reader, Scanner, and any combinations thereof as known to people who are skilled in the art. Moreover, performing the steps from adding the molecular beacon to observing the result takes no more than 2 hours.

Another aspect of the invention relates to a method for detecting a cell having an infectious disease, e.g., detecting a flu virus-infected cell. The flu virus includes an avian flu virus, e.g., fluA, and fluB viruses. The fluA virus may be one of 16H and 9N strains, and any combinations thereof. For example, the cell to be detected by the method of the invention may be infected by a flu virus that is one of fluA, fluAH5, fluA N1, and any combinations thereof.

Yet another embodiment of the invention is a method for detecting a cancer cell in a specimen containing one or more cells. The cancer cell may be lung cancer, liver cancer, stomach cancer, prostate cancer, breast cancer, pancreatic cancer, skin cancer, bone cancer, womb cancer, cervical cancer, brain cancer, or colon cancer.

Another embodiment of the invention is a method for detecting at least one point mutation and/or deletion in a specific marker of a cancer cell.

Yet another embodiment of the invention is a method for detecting a mutation, either a point mutation and/or deletion, of a disease marker. The disease marker includes a biological target of a targeted therapeutics. The biological target includes EGFR gene.

One embodiment of the invention is a method for detecting a cancer cell marker in a specimen, in which the cancer cell marker contains a deletion mutation in EGFR tyrosine kinase domain.

Yet another embodiment of the invention is a method in which one or more probes are added into the cell specimen.

Another aspect of the invention provides a molecular beacon that is a single stranded hairpin shaped structured oligonucleotide probe containing a nucleotide sequence selected from the group consisting of SEQ ID NOs: 1-11, and any combinations thereof. The molecular beacon is capable of hybridizing with a disease-related RNA or DNA of a disease marker in at least one cell in a specimen from a living subject, thereby emitting a signal detectable without a need for signal amplification.

One embodiment of the invention is a MB containing a nucleotide sequence capable of hybridizing with RNA and/or DNA that encodes a universal cancer marker. In one embodiment of the invention, the molecular beacon is capable of hybridizing with a transcription product of EGFR.

Another embodiment of the invention is a MB that contains a nucleotide sequence capable of hybridizing with RNA and/or DNA encoding EGFR gene tyrosine kinase domain in a cancer cell. The MB comprises a fluorofore at 5′ and a quencher at 3′, or a fluorofore at 3′ and a quencher at 5′.

Yet another embodiment of the invention is a molecular beacon that is capable of detecting a drug-resistant cancer and/or a drug-resistant pathogen.

Yet another aspect of the invention is a diagnostic kit for detecting an infection and/or expression or a mutation of a disease marker for diagnostics and pharmacogenomics in a living subject, in which the diagnostic kit contains a molecular beacon of the invention and an instruction sheet. In one embodiment, the diagnostic kit includes a MB containing a nucleotide sequence selected from the group consisting of SEQ ID NOs: 1-11. The MB and the assay method described in the instruction sheet in the diagnostic kit enables a performance of diagnosis, from adding the molecular beacon into a specimen to observing a result therefrom, taking no more than 2 hours.

The accompanying drawings illustrate one or more embodiments of the invention and, together with the written description, serve to explain the principles of the invention. Wherever possible, the same reference numbers are used throughout the drawings to refer to the same or like elements of an embodiment.

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1

Molecular Beacon-Target DNA Fluorescence Testing: The method for measuring binding of molecular beacons to DNA template (measurement of MB specificity) is described as follows:

Materials includes: Opti-MEM Transfection Solution (Invitrogen), Costar 96-well black plates (eBioscience Catalog No. 44-2504-21), 1.7 mL Eppendorf tubes (Denville Catalog No. C-2170), Standard PCR Tubes, Molecular Beacons (MWG-Biotech AG), and Target DNA (MWG-Biotech AG).

The procedure is as follows:

(1) Diluting of Molecular Beacons and Target DNA: Based on MWG Oligo Synthesis Report, dilute the molecular beacons and target DNA according to the amount of transfection solution specified by the “Volume for 100 pmol/μl.” Vortex and spin. Aliquot an equal amount of an oligo solution and place in −20° C. freezer away from light.

(2) Preparation of Fluorescence Testing: Dilute each molecular beacon with a 1:10 dilution (1 μl of molecular beacon solution with 9 μl of transfection solution). Make two fluorescence mixes for each molecular beacon—a target-to-DNA mix and a control mix with 1 μl of DNA, 2 μl of molecular beacon 1:10 dilution, and 97 μl of transfection solution. Allow to incubate away from light at 37° C. for one hour.

(3) Running the test and getting the results. Place 95 μl of each mix into a well in a 96-well black plate. Run plate in SpectraMAX GeminiXS (from Molecular Devices) fluorescence machine and the SoftMAX Pro 4.3.1 LS Software using the following settings: Click “setup.” Settings are set at “Endpoint,” and Read Type is “Fluorescence (RFU's).” Change “Number of wavelengths” to 3, and follow the table below:

TABLE 1
Fluorescence and wavelengths of the MB-target DNA
fluorescence testing.

| Fluorescence Name | Excitation Wavelength | Emission Wavelength |
|---|---|---|
| Texas Red | 590 | 615 |
| FAM | 488 | 515 |
| CY3 | 530 | 575 |

Go to “Sensitivity” and drag the number of readings to 8. Go to “Wells to Read” and select the wells in which you want to read. Click “Read.” Go to “File” and “Import/Export.” Export the results as a text document onto a floppy drive. Results are read from a floppy drive using MICROSOFT® Excel.

### Example 2

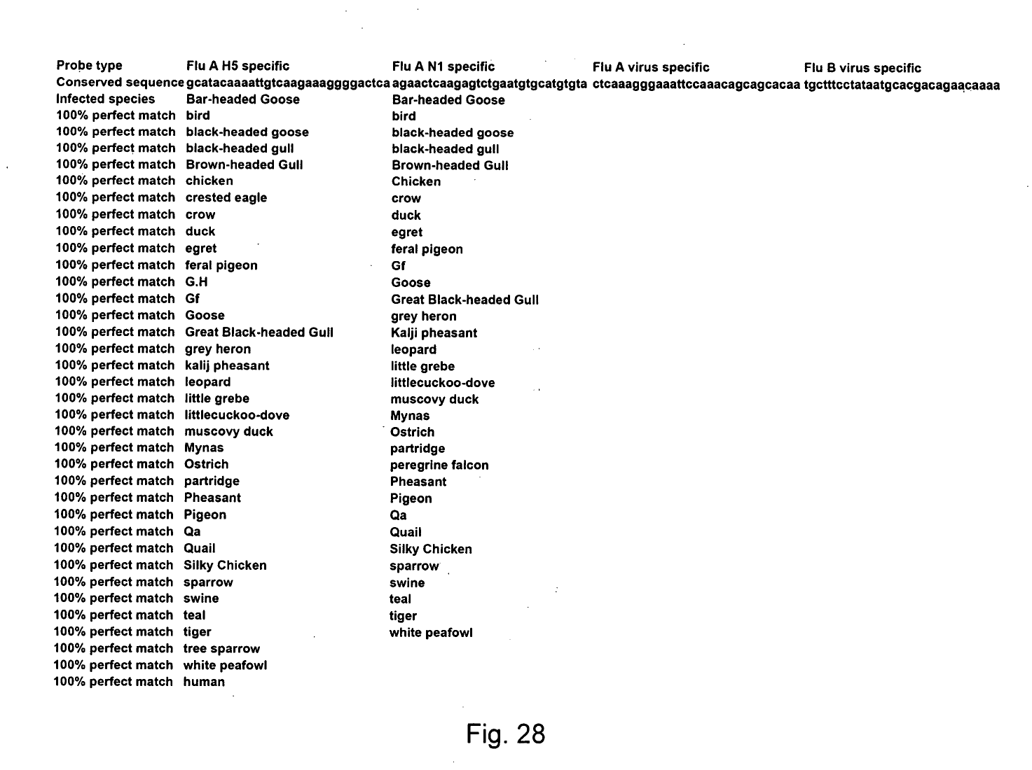

Molecular beacons (MBs) for detecting FluA, FluB, FluAH5 and FluAN1, as shown in Table 2, were designed based on the specific DNA sequences identified by bioinformatics, respectively. The formation of hairpin loop was designed to have 5 or 6 (most of time 5) base pairs. A general method for making a MB is disclosed by Peng et. al. (18). The MBs were then synthesized by a contractor MWG Biotech, Inc. located in North Carolina. The 5′ (or 3′) fluorofores can be any other fluorescent proteins, and the quenchers at the 3′ (or 5′) can be any other quenchers that can quench the corresponding florescent group. FIG. 28 shows conserved sequences identified by bioinforrnatics that are specific to flu virus types of FluA and FluB, and strains of FluAH5 and FluAN1.

As shown in Table 3, sequences identified by bioinformatics that are specific to flu virus types of FluA and FluB, and strains of FluAH5 and FluAN1.

TABLE 2
Molecular beacons and SEQ ID NOs

| SEQ | | |
|---|---|---|
| ID NO: | Nucleotide Sequence | Oligo Name |
| 8 | 5′CY3-CTGAGTCCCCTTTCTTGACCTCAG- | ALVFLUAH5MB |
| | 3′BHQ2 | |
| 9 | 5′FAM-CACACATGCACATTCAGACGTGTG- | ALVFLUAN1MB |
| | 3′BHQ1 | |
| 10 | 5′CY3-CGTGCTGCTGTTTGGAATTGCACG- | ALVFLUAMB |
| | 3′BHQ2 | |
| 11 | 5′FAM-CGTTCTGTCGTGCATTATAGGAACG- | ALVFLUBMB |
| | 3′BHQ1 | |

Black Hole Quencher (BHQ ®) dyes

TABLE 3

sequences specific to FluA and FluB, and strains
of FluAH5 and FluAN1

| SEQ | | |
|---|---|---|
| ID | | Flu |
| NO: | Nucleotide Sequence | Virus Type |
| 12 | GCATACAAAATTGTCAAGAAAGGGGACTCA | Flu A H5 |
| | | specific |
| 13 | AGAACTCAAGAGTCTGAATGTGCATGTGTA | Flu A N1 |
| | | specific |
| 14 | CTCAAAGGGAAATTCCAAACAGCAGCACAA | Flu A virus |
| | | specific |
| 15 | TGCTTTCCTATAATGCACGACAGAACAAAA | Flu B virus |
| | | specific |

### Example 3

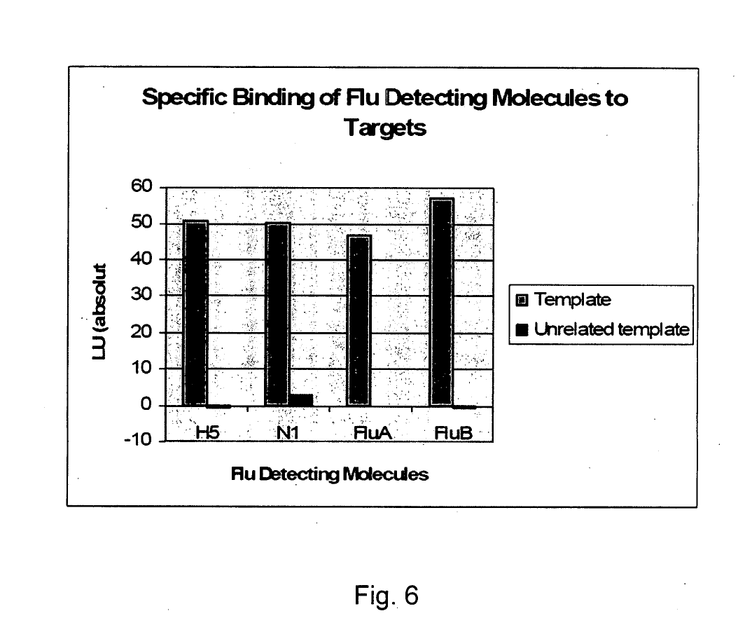

Infectious Disease Detection: The flu-detecting molecules of the present invention showed specific binding to targets. Molecules such as ALV-Flu A, ALV-Flu A H5, ALV-Flu A N1 and ALV-Flu B were designed to specifically detect Flu A, Flu A H5, Flu A N1 and Flu B, respectively. As shown in FIG. 6, these molecules specifically bind to their respective targets with very low background.

### Example 4

Method for Rapid Testing of Flu Virus Infection in Cell Cultures: Materials includes: Cell Culture Slides 25×75×1 mm (VWR Cat. No. 48312-400); Slide Cover Slips 22×50 mm No 1½ (VWR Cat # 48383 194); Dako Pen (Cat. No. S2002); Cell Culture Media (RPMI-1640); Opti-MEM Transfection Solution (Invitrogen); 0.25% Trypsin EDTA Solution (Invitrogen); Gel/Mount (Biomeda Corp. Cat. No. M01); Hoechst 33342 (Cambrex, Cat. No. PA-3014); Triton X-100 (Merck); and molecular beacons reagents for FluA, FluB, FluAH5 and FluAN1 100 uM stocks in Opti-MEM.

The Procedure is as follows:

(1) Fixing cells on slides: Label slides with pencil, as acetone would dissolve black ink. Then wash slides once with serum free culture medium, and once with sterile PBS. Afterwards, soak the slides in ice cold 100% acetone for 8-10 minutes. Allow slides to air dry. If slides will not be used immediately, place slides in −80° C. for storage.

(2) Triton Treatment: Wash slides once with ice cold serum free culture medium, and once with ice cold sterile PBS. Then Soak in 0.2% Triton solution in PBS at 37° C. for 20 minutes, and wash twice with ice cold PBS.

(3) Adding MB detecting reagents of the present invention: Draw circles around the wells on slides with Dako Pen. Then make an appropriate concentration from 100 uM stocks of molecular beacons reagents with Opti-MEM, e.g. 300 nM. Afterwards, add 100 μl (25-35 ul for 8-well slide) of MB reagents of the present invention to appropriate circles on cell slides, and place in 37° C. incubator with humility for 20 minutes.

(4) Staining nuclei of the cells: After incubation for 20 minutes, remove the solution from slides. For fluorescent microscope, add Hoechst 33342 (1/1000 dilution of 10 mg/ml stock in PBS) to each cell circle. Place in a 37° C. incubator for no more than 2-4 minutes.

(5) Finishing: Remove slides from the incubator. Then wash slides twice with ice cold sterile PBS. If for fluorescent microscope, add one drop of slide gel/mount to each cell circle. Place a cover slip over the slide.

(6) Observation of results under a fluorescence microscope (Olympus DP70): To the left of the microscope, turn on the fluorescence power supply. On the right side of the microscope, turn on the power to the microscope. Place the slide under the fluorescent microscope and locate cells using the DAPI filter for Hoechst 33342. Once locate some cells, switch between different fluorescent light to find appropriate beacon fluorescence. When ready to take a picture, go to the computer and double-click on the “DPControllers” icon. Use the following settings for each fluorescent light:

TABLE 4
Testing dada sheet.

| Molecular Beacon | Fluorescent Setting |
|---|---|
| Texas Red | Rhodamine |
| CY3 | Rhodamine |
| FAM | FITC |
| White Light | DAPI |

First, click “Snap” to capture the picture onto the computer. Then click “Save as” in order to save the picture onto the appropriate file in computer. Take a picture of the cells under their appropriate fluorescent light as well as DAPI to make sure cells are present in the picture. Once finished, turn off the fluorescence power supply, fluorescence microscope and computer.

### Example 5

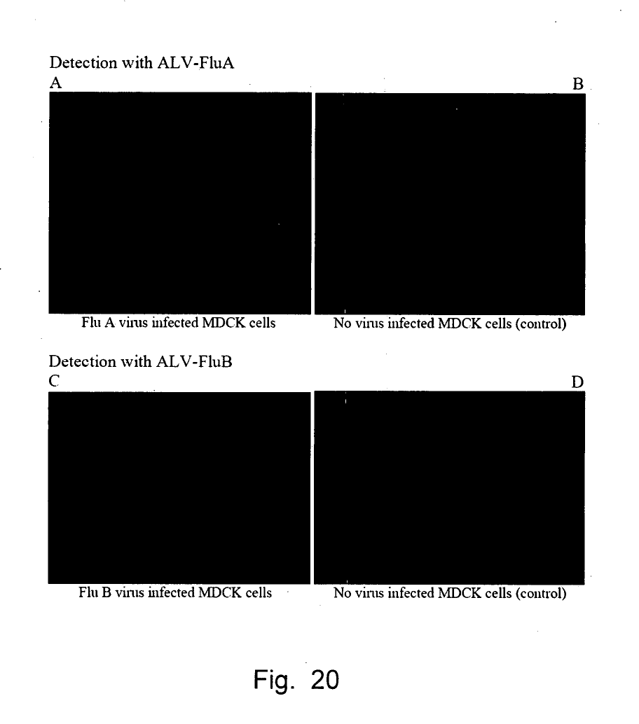

Preclinical Studies for Detection of Flu Virus Infection in Cell Culture: Briefly, cell culture of dog kidney epithelial cells, MDCK, after infected with A or B subtype flu viruses for two to three days were stained with molecular beacons specific to flu A (ALV-FluA) and flu B (ALV-FluB), respectively. After completion of the 20-minute staining, the cells attached to slides were analyzed under a fluorescence microscope. As shown in FIG. 20, cells infected by flu A virus were detected specifically by the flu A detection product ALV-FluA (red color in panel A), while cells infected by flu B virus were detected by the product for flu B virus infection ALV-FluB (green color in panel C).

### Example 6

Clinical Studies for Detection of Flu Virus Infection in Patients: During the winter flu season of 2005-2006, a clinical study was designed under IRB guidelines in collaboration with a leading university hospital in Asia to evaluate feasibility of using molecular beacons of the present invention for rapid detection of flu infection. As a standard procedure, throat swabs from patients were smeared as samples collected on microscope slides. Separate swab samples were also collected for viral culture and RNA extraction for RT-PCR analyses. The slides were detected with a molecular beacon flu product containing a mixture of ALV-FluA and ALV-FluB specific to flu A and flu B viruses, respectively, or corresponding control reagents for red and green fluorescence ALV-RanRed and ALV-RanGreen. The results from this blind pivotal clinical study were very successful in that detection with the molecular beacon products was more than 90% consistent with those obtained from RT-PCR.

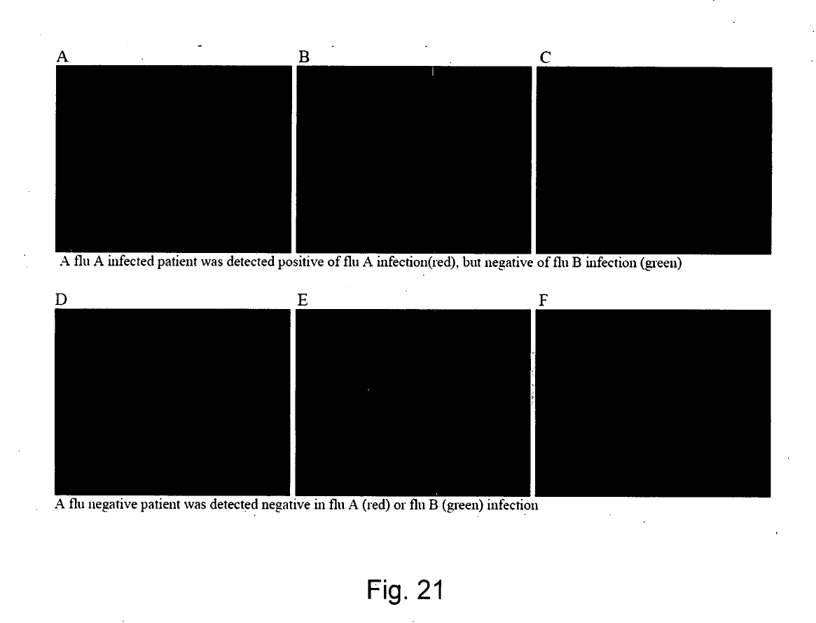

In the representative result as shown in FIG. 21, a patient infected by the flu A virus as confirmed by RT-PCR was detected with a product containing mixed reagents ALV-FluA and ALV-FluB specific to flu A and flu B viruses, respectively. As shown in FIG. 21, the patient was detected as positive for flu A virus infection (red in panel A) but not flu B virus (green in panel B). Another patient who was free of flu virus infection was detected negative by ALV-FluA (red color in panel D) and ALV-FluB (green color in panel E). The blue fluorescence in panel C and F was the staining of nuclei of corresponding cells.

### Example 7

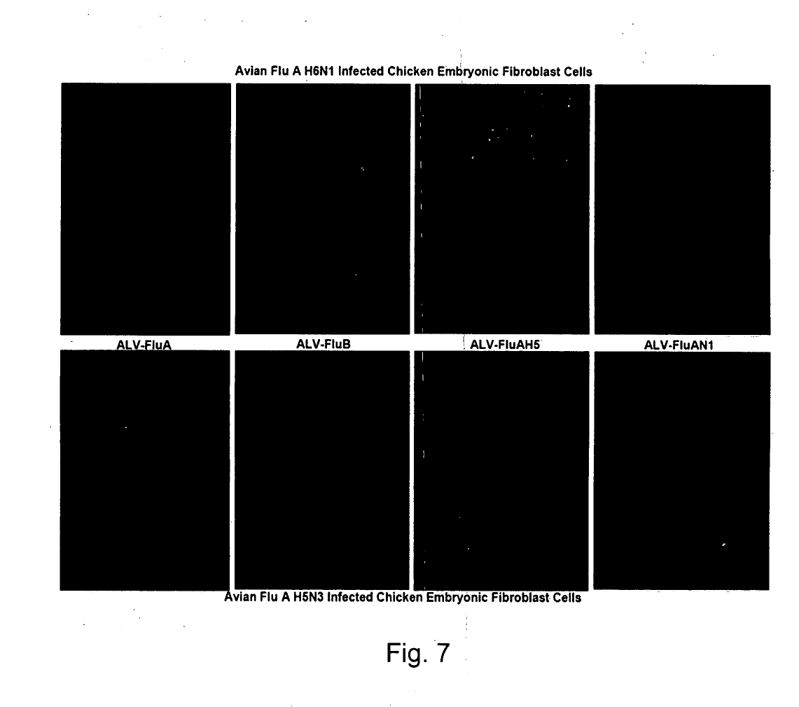

Reagents detecting Infection of Avian Flu Virus were developed. Assays using the designed Flu detecting molecules specific for Flu A, Flu A H5, Flu A N1 and Flu B were developed for rapid and sensitive detection of Flu A (H5N1 and HH6N1) infection. Upon infection, the infected host was rapidly detected using detection agents of the present invention. As shown in FIG. 7, the host infected by the avian Flu A (H6N1) virus was identified using molecular beacons of the present invention, ALV-Flu A (for Flu A, red) and ALV-Flu A N1 (for N1, green), respectively. Similarly, host infected by avian Flu A (H5N3) virus was identified using molecular beacons of the present invention ALV-Flu A (for Flu A, red) and ALV-Flu A H5 (for Flu A H5), respectively.

### Example 8

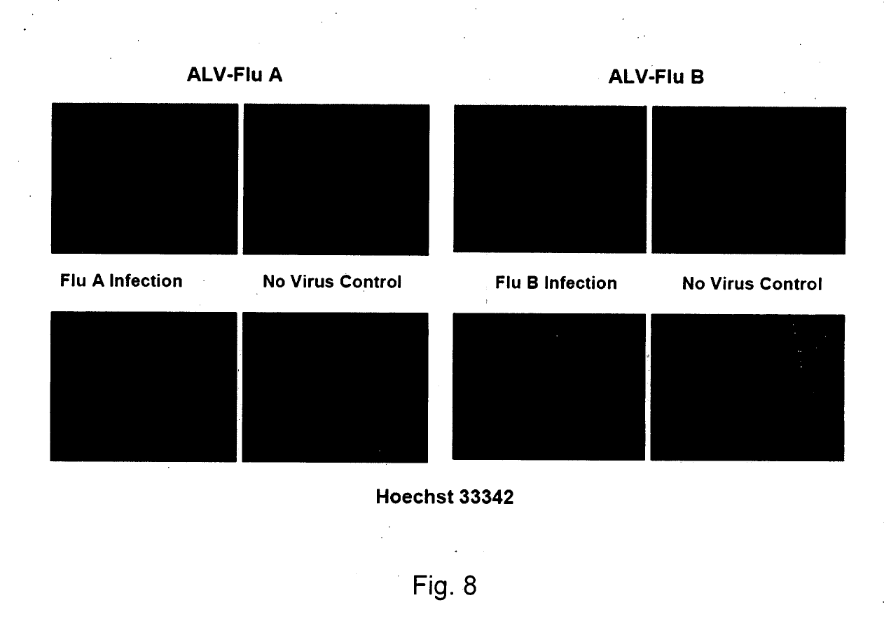

Test agents were developed for detection of both human and avian flu virus infections. The detection molecules ALV-Flu A and ALV-Flu B were specific to flu virus A and B, respectively. They are able to detect infections in human that are caused by flu virus strains A and B. As shown in FIG. 8, the results demonstrated that ALV-Flu A and ALV-Flu B detected Flu A and Flu B virus infection specifically.

### Example 9

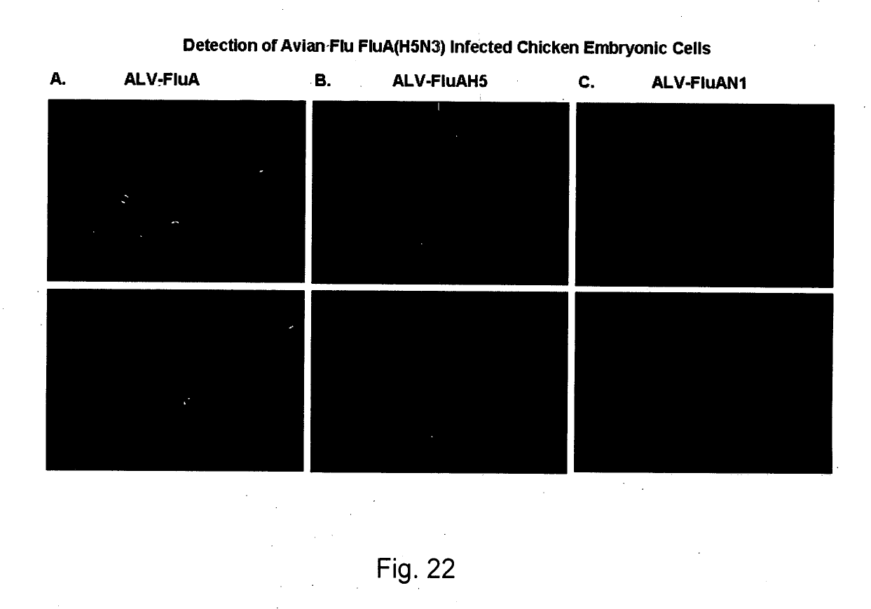

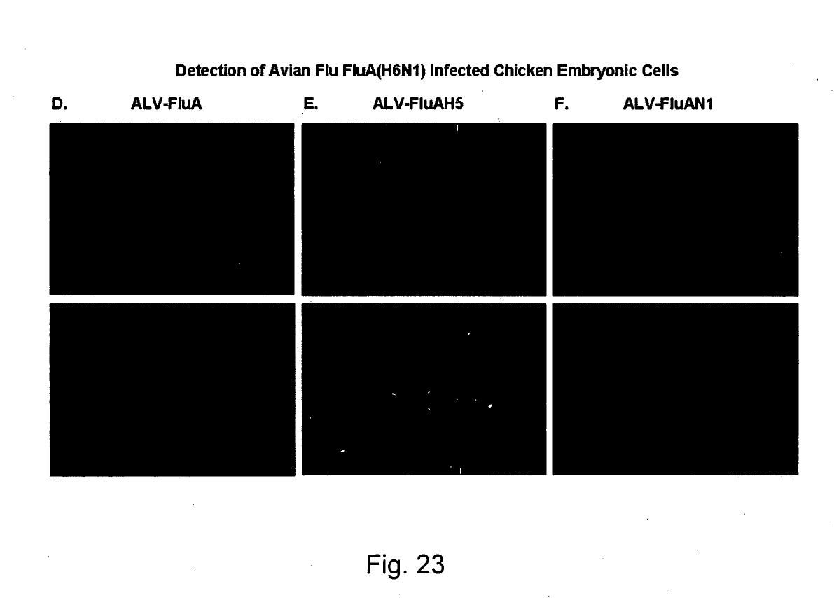

Detection of Avian Flu Virus H5 and N1 Infections: In addition to the product ALV-FluA for detection of pan flu A virus infection, ALV-FluAH5 and ALV-FluAN1 products were specific to flu A(H5) and flu A(N1) virus strains, respectively. In combination with ALV-FluA, ALV-FluAH5 and ALV-FluAN1 reagents, assays using the product should be specific for detection of flu A(H5N1) infection. However, due to the limited access and potential severe hazards of flu A(H5N1) infected human or animal specimens, the studies to evaluate feasibility of using ALV-FluAH5 and ALV-FluAN1 for detection of flu A(H5) and flu A(N1) virus infections were carried out with flu A(H5N3) and flu A(H6N1) infected chicken embryonic cells. Flu A(H5N3) infected cells served as the model for flu A(H5) detection and flu A(H6N1) for flu A(N1). With the model systems, in which chicken embryonic cells were infected with avian flu A(H5N3) or flu A(H6N1), the infected host cells were detected with molecular beacons products of the present invention. As shown in FIG. 22, the host cells infected by the avian flu A(H5N3) virus were specifically identified using ALV-FluA (for flu A, red in panel A) and ALV-FluAH5 (for flu A(H5) red in panel B), respectively. Similarly, as shown in FIG. 23, the host cells infected by avian flu A(H6N1) virus were identified using molecular beacons of the present invention, ALV-FluA (for flu A, red in panel D) and ALV-FluAN1 (for flu A(N1), green in panel F), respectively. The blue fluorescence was the staining of nuclei of each corresponding cell culture.

Key features for ultra-sensitive molecular detection (USDM) plateform technology include: (1) an innovation of rapid and powerful technology to detect expression and mutation of genes of interest; (2) suitable for early detection of disease progression and pharmacogenomics, (3) one-step assay with final signal read out in 10-20 minutes.

Molecular beacon products of the present invention are sensitivity for detection of Avian Flu Virus Infection. The present invention provides detecting molecules that are specific to Flu A, Flu B, Flu A H5 and Flu A N1. Molecules for detection of avian flu infection include: ALV-FluA—red color, ALV-FluB—green color, ALV-FluA H5—red color, and ALV-FluA N1—green color.

Hoechst 33342—DNA staining for cells shows in blue color. These molecular beacon products of the present invention were designed to detect infection of flu viruses from various species. Animals where avian flu virus can be detected include bird, chicken, duck, goose, pigeon, swine, human, etc.

### Example 10

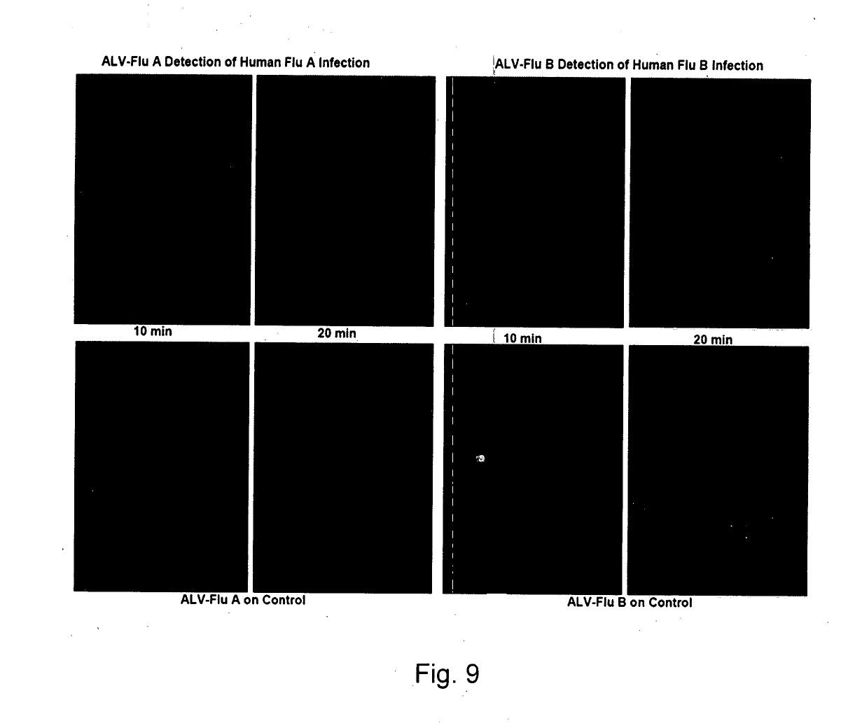

The detection method of the present invention has proved to be a rapid one-step assay with high fidelity. The MB-based detection of flu virus infection according to one embodiment of the present invention is a simple one-step assay. The whole process takes only 10 to 20 minutes. As shown in FIG. 9, the assay gave very low or no background at 10 or 20 minutes when the human Flu A or Flu B virus infection was detected.

### Example 11

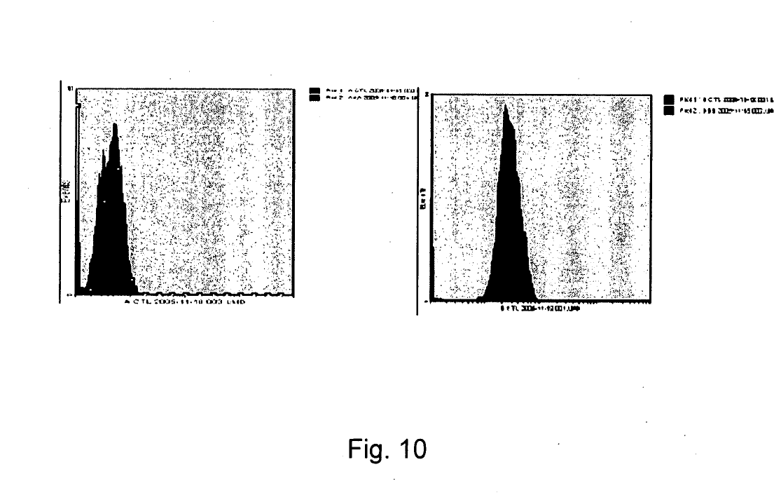

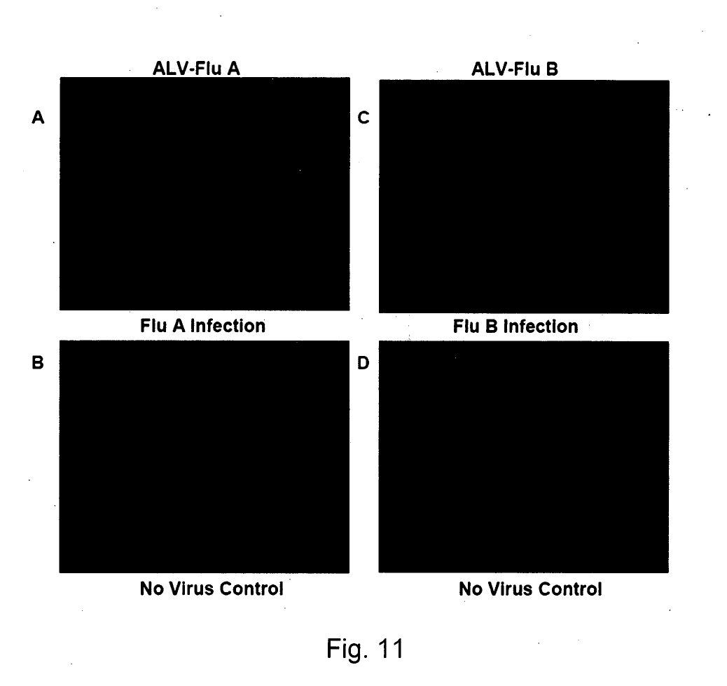

The assay results from the use of the molecular beacons of the invention can be easily handled. For example, the results generated from assays of the present invention for infection of flu viruses can be measured with instruments commonly used in the clinical sites. In addition to the fluorescent microscope applied with the results as shown previously, the assay can also be measured with Fluorescent Activated Cell Sorter (FACS), a machine being routinely utilized to measure the white blood cell counts in HIV infected patients. FIG. 10 is a typical quantitative histogram showing the ALV-Flu A and ALV-Flu B detection of human Flu A and Flu B virus infection. The FACS result is very consistent with what is obtained using fluorescent microscope as shown in FIG. 11. Other routine methods for readout of assay results are in the process of being evaluated.

The detection molecule of the present invention showed a quick response to the outbreak of drug-resistant strains. Like in the cancer pharmacogenomics, flu virus-detecting molecules of the present invention are able to detect mutations including point mutations and deletions. Should the outbreak of drug, e.g. Tamiflu,-resistant strain of avian flu virus occurs, the turn around time required for molecular design and production of detection molecule(s) of the present invention is in the range of 2-3 weeks once the mutated sequences are identified. That is incomparable to assays based on development of antibodies.

The detection molecule of the present invention may be expanded to cover wide spectrum of avian flu strains including 16 H and 9N strains; and turn around quickly with readiness in response to the occurrence of drug resistant strain outbreak.

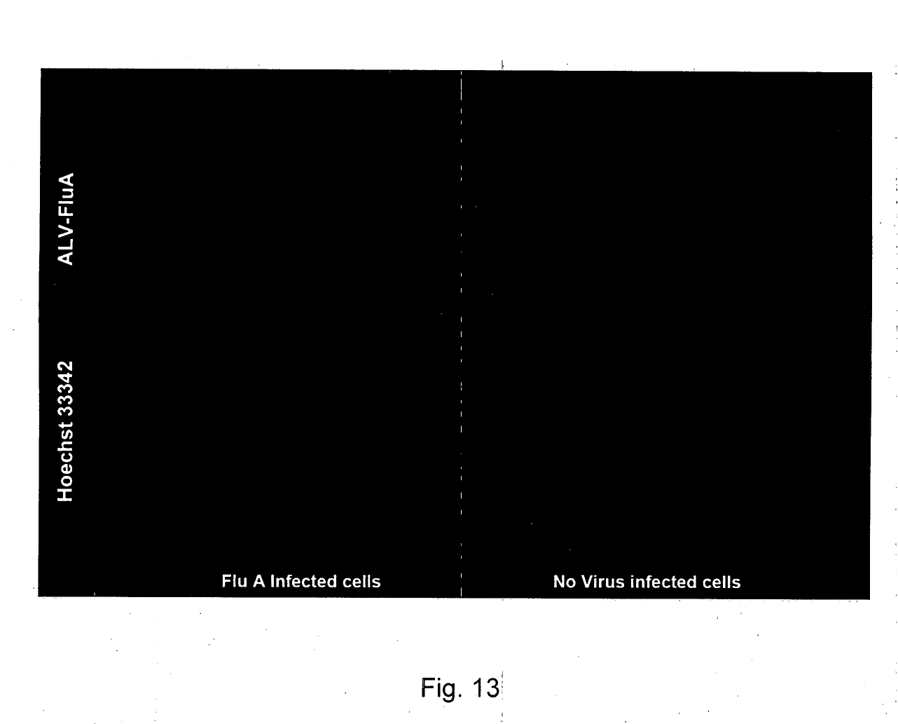

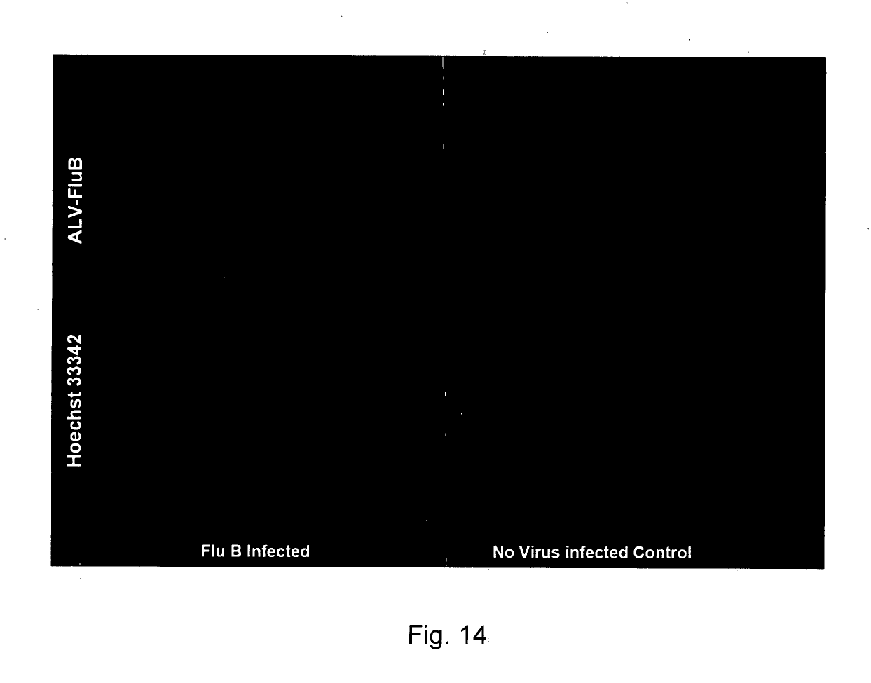

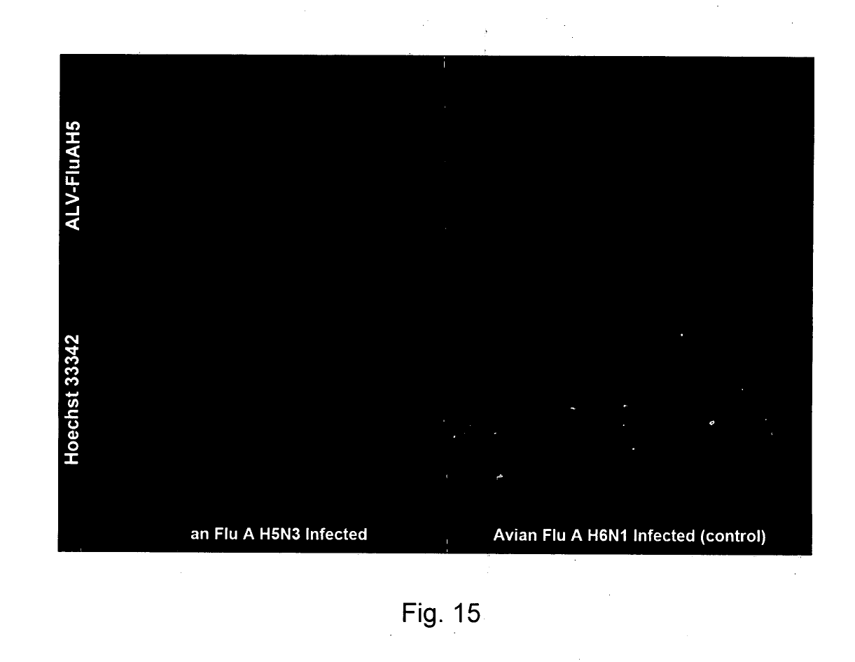

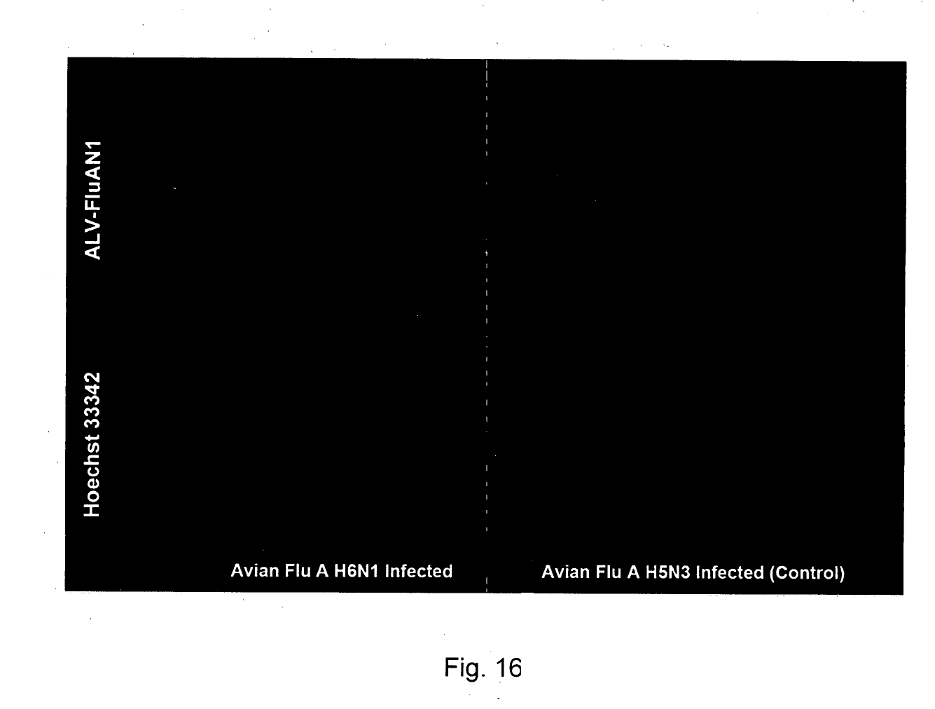

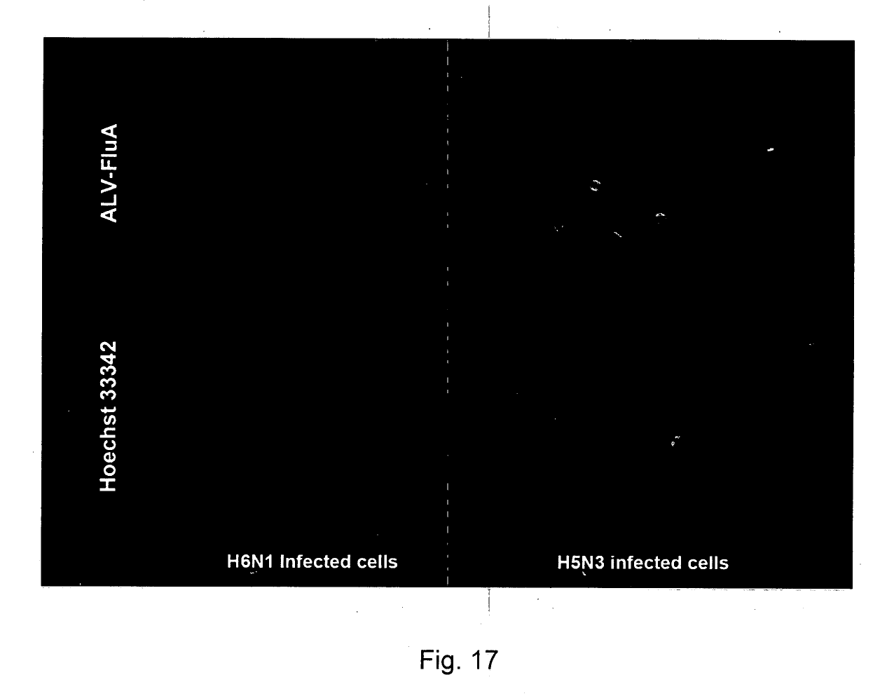

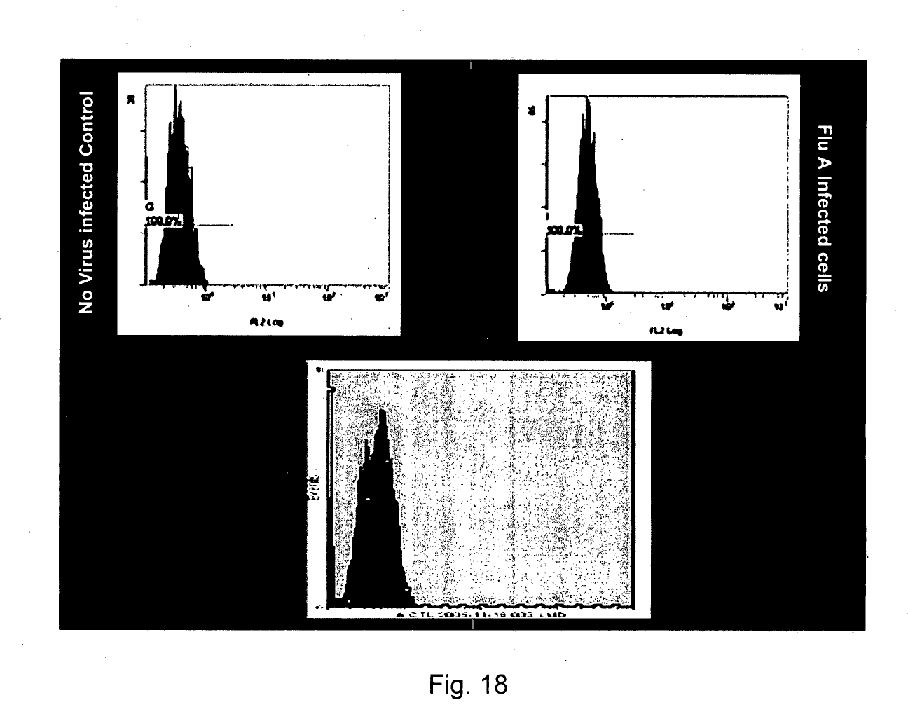

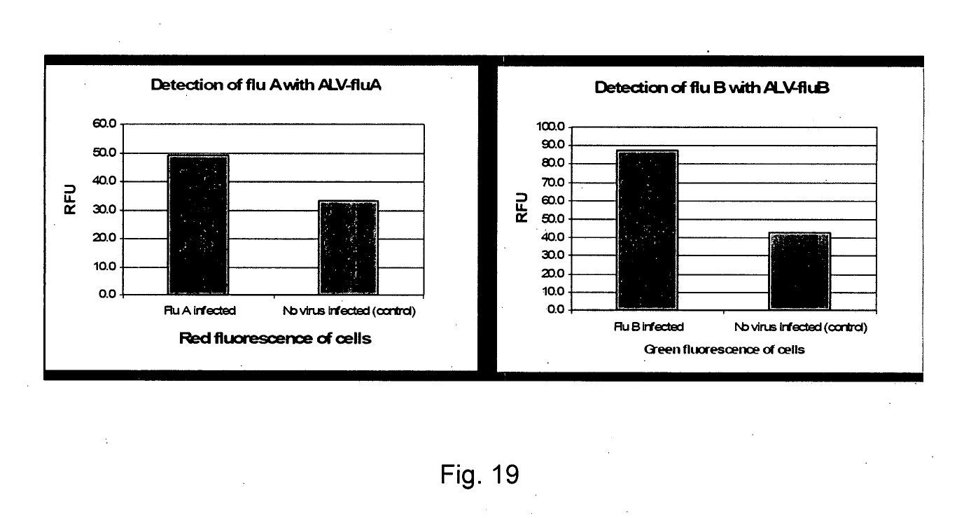

FIGS. 13-19 show the detection molecules of the present invention: ALV-Flu A detection of human Flu A virus infection (FIG. 13), ALV-Flu B detection of human Flu B virus infection (FIG. 14), ALV-Flu H5 detection of human Flu H5 virus infection (FIG. 15), ALV-Flu AN1 detection of Avian Flu A N1 virus infection (FIG. 16), ALV-Flu A detection of Avian Flu A virus infection (FIG. 17), FACS analysis of Flu virus infection following ALV-Flu A detection (FIG. 18), RFU analysis of human Flu virus infection with fluorescence plate reade (FIG. 19).

TABLE 5
Simplicity of the MBs of the present invention signal read
out using instruments common to clinical laboratories

| | | | | Popularity in |
|---|---|---|---|---|
| | Measurement | Speed | Cost | Clinical Lab |
| Microscope | Visual | +++++ | Low | Very common |
| | Single cell | | | |
| | Qualitative | | | |
| Flow | Visual | ++ | High | Common in |
| Cytometry | Single cell | | | AIDS |
| | Qualitative | | | |
| | Percent population | | | |
| Microplate | Light units | +++++ | Low | Very common |
| Reader | Total cell signals | | | |
| | Quantitative | | | |

In summary, the detection molecule of the present invention is a highly sensitive agent for detection of flu virus infection, including avian flu infection. For example, ALV-FluA and ALV-FluB are sensitive for differentiating human flu A and B subtypes and ALV-FluAH5 and ALV-FluAN1 for detecting flu A(H5) and flu A(N1) avian flu strains. Moreover, in combination with ALV-FluAH5, ALV-FluAN1 and ALV-FluA have the potential of rapidly detecting infection of flu A(H5N1) strain. Furthermore, the detection molecule of the present invention is a rapid one-step assay and takes only 10, 20, or 30 minutes or less for the assay process. Analysis of detection signal read out flexible and simple. These detection molecules of the invention have the possibility for expansion to detect a wide spectrum of flu strains including potential deadly strains in the 16H and 9N families.

### Example 12

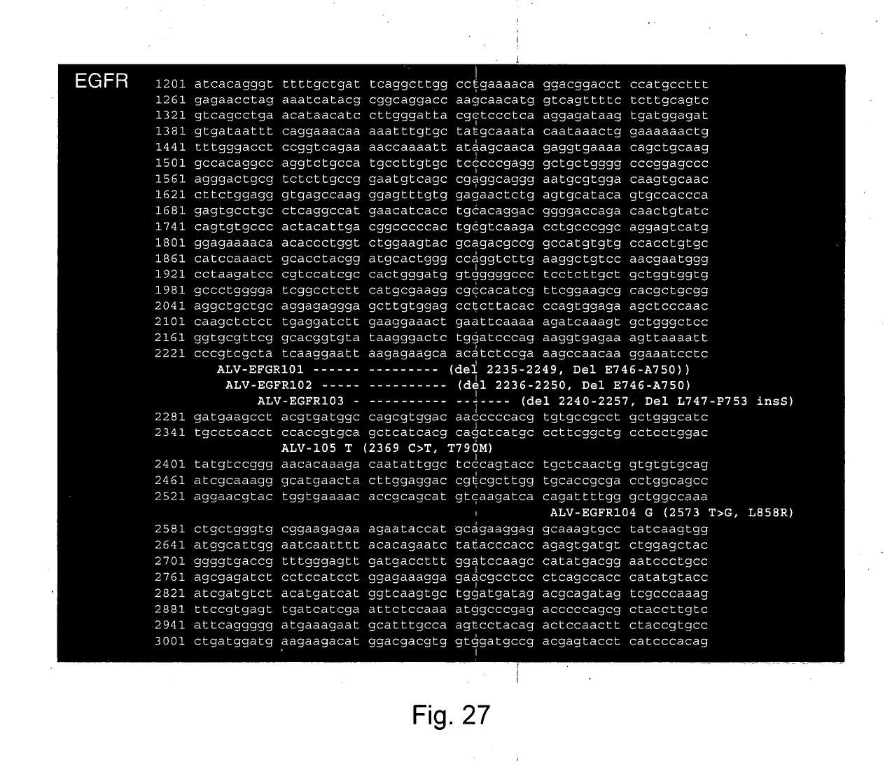

Cancer Marker Detection: Table 6 shows molecular beacons for detection of EGFR point mutations and deletions and MB for detecting surviving as positive control and random as negative control. Fluorofore at 5′(or 3′) and quencher at 3′(or 5′) can be any other fluorofors or quenchers, as long as they can be quenched. For ALV-EGFR 101˜105, their corresponding position in the EGFR gene is shown in the FIG. 27.

TABLE 6
Nucleotide Sequences

| SEQ | | |
|---|---|---|
| ID | | Oligo |
| NO: | Nucleotide Sequence | Name |
| 1 | 5′RED-TCGCTGCTTTCGGAGATGTTTTGATAGCGA- | AEGFR1 |
| | 3′BHQ1 | 01 |
| 2 | 5′RED-TCGCTGCTTTCGGAGAATGTCTTGATAGCGA- | AEGFR1 |
| | 3′BHQ1 | 02 |
| 3 | 5′RED-TCGCTGGCTTTCGATTCCTTGATAGCGA- | AEGFR1 |
| | 3′BHQ1 | 03 |
| 4 | 5′CY3-CAGATTGGCCCGCCCAAAATCTG-3′BHQ1 | AEGFR1 |
| | | 04 |
| 5 | 5′FAM-TGCAGGCATGAGCTGCATGATGAGCTGCA- | AEGFR1 |
| | 3′BHQ1 | 05 |
| 6 | 5′CY3-CACGTCGACAAGCGACCGATACGTG-3′BHQ1 | ARAND |
| | | OMR01 |
| 7 | 5′FAM-TGGTCCTTGAGAAAGGGCGACCA-3′BHQ1 | ASURVI |
| | | VINC01 |

### Example 13

Lung Cancer Cell Testing With Molecular Beacon Reagents: The following is the method that was used for detection of EGFR point mutation and/or deletion for cancer pharmacogenomics. EGFR, an abbreviation of epidermal growth factor receptor, is a protein found on the surface of cells to which epidermal growth factor (EGF) binds.

Materials includes: Cell Culture Slides 25×75×1 mm (VWR Cat. No. 48312-400), Dako Pen (Cat. No. S2002), Cell Culture Media (RPMI-1640), Opti-MEM Transfection Solution (Invitrogen), 0.25% Trypsin EDTA Solution, Gel/Mount (Biomeda Corp. Cat. No. M01), and Hoechst 33342.

The Procedure is a follows:

Washing and Coating slides (this is done only if cells do not attach well): Soak slides in 70% Ethanol at Room Temperature for 30 minutes. (Fluorescent Antibody Rite-On Micro Slides, One end frosted, 2 etched rings, Size 3×1′, Thickness 0.93-1.05 mm, ˜0.5 Gross. Gold Seal Cat #3032). Remove slides from ethanol and let air dry. Coat one side of the slides with sterile (by autoclave) 1% Gelatin (in H₂O) for 1 hour at room temperature. Remove the Gelatin solution and let slides air dry.

Fixing Cell Line onto slides: Draw two large circles (with DAKO pen) on the slides to distinguish where the cell lines will be placed. (Dako Pen, Cat. # S2002). Spin down cells in lung fluid samples collected from cancer patients. Resuspend the cells in serum free cell culture medium to the density of ˜10⁶ cells/ml. Drop two to three drops of cells in culture media to the appropriate slides. Place slides on a tray for convenience of handling. Place tray in incubator chamber and into the 37° C. incubator with 2% CO₂ for 2-4 hours or until most of the cells have attached. Wash slides 1× with serum free culture medium, 1× with sterile PBS. Soak the plates in ice cold 100% acetone for 8-10 minutes. Label slides with pencil as acetone will dissolve black ink. Let slides air dry. If slides will not be used immediately, store slides in −80° C.

Adding the MB reagents: Wash slides 1 × with serum free culture medium, 1× with sterile PBS. Make appropriate concentration from 100 uM stocks of MB reagents in serum free medium as needed, e.g. 200 nM and 50 nM. Add 100 μl of MB reagent solution to appropriate circles on cell slides. Place in 37° C. incubator for about one hour.

Staining the cells: After incubation for an hour, wash slides 2× with sterile PBS. Add the Hoechst 33342 (1/1000 dilution of 10 mg/ml stock in PBS) to each cell circle. Place in 37° C. incubator for no more than 2-3 minutes.

Finishing: Remove slides from incubator. Wash slides 2× with sterile PBS. Add one drop of slide gel/mount to each cell circle. Place a cover slip over each circle. (VWR micro cover glass 22×50 mm, No. 1½, VWR Cat #48393 194)

Fluorescence Testing under the fluorescent microscope (Zeiss Axioplan 2): To the right of the microscope, turn on the fluorescence power supply. On the right side of the microscope, turn on power to the microscope. Connect the black cable to the back of the blue AxioCam HRc on top of the microscope. Place slide under fluorescent microscope and locate cells using the white light filter. Once you locate some cells, you can switch between different fluorescent light to find appropriate beacon fluorescence. When you are ready to take a picture, go to the computer and double-click on the “AxioVision 4” icon. On the side toolbar, open the AxioCamHR Control. Use the following settings for each fluorescent light: Set Exposure percent should be set at 80%.

TABLE 7
Testing dada sheet.

| Molecular Beacon | Fluorescent Setting | Exposure Time |
|---|---|---|
| Texas Red | Rhodamine | 486 ms |
| CY3 | Rhodamine | 486 ms |
| FAM | FITC | 1.1 s |
| White Light | DAPI | 5 ms |

Open the camera window on the right side of the microscope. Click “Live” to view a live picture of the slide on the computer. Click “Snap” to capture the picture onto the computer. Click “Export” in order to save the picture onto the computer. Make sure to take a picture of the cells under their appropriate fluorescent light as well as white light to make sure cells are present in the picture. Once finished, make sure to turn off the fluorescent microscope.

### Example 14

Detecting EGFR Mutations in Lung Cancer: About 40% of patients with non-small cell lung cancer (NSCLC) are found to have specific mutations in the epithelial growth factor receptor (EGFR) gene. The mutations and/or deletions in EGFR are believed to correlate with clinical responsiveness to the tyrosine kinase inhibitor, e.g. gefitinib (Irressa) and erlotinib (Tarceva). These mutations lead to increased growth factor signaling and confer susceptibility to inhibitor therapeutics. Screening for such mutations in lung cancer may identify patients who will have a better response rate to the targeted therapy. Development of novel approaches for early screening of cancer patients is of critical importance for the successful treatment and for increasing survival of the patients.

The initial focus in cancer was to develop and commercialize the diagnostic and pharmacogenomic products based on MB technology to improve therapeutic efficacy of medicines targeted to EGFR—its mutations affecting downstream signaling has direct impacts on response and survival in cancer patients treated with therapeutics targeted to EGFR. The products of the invention cover more than 80% of the EGFR mutations commonly found affecting response to EGFR targeted medicines.

### Example 15

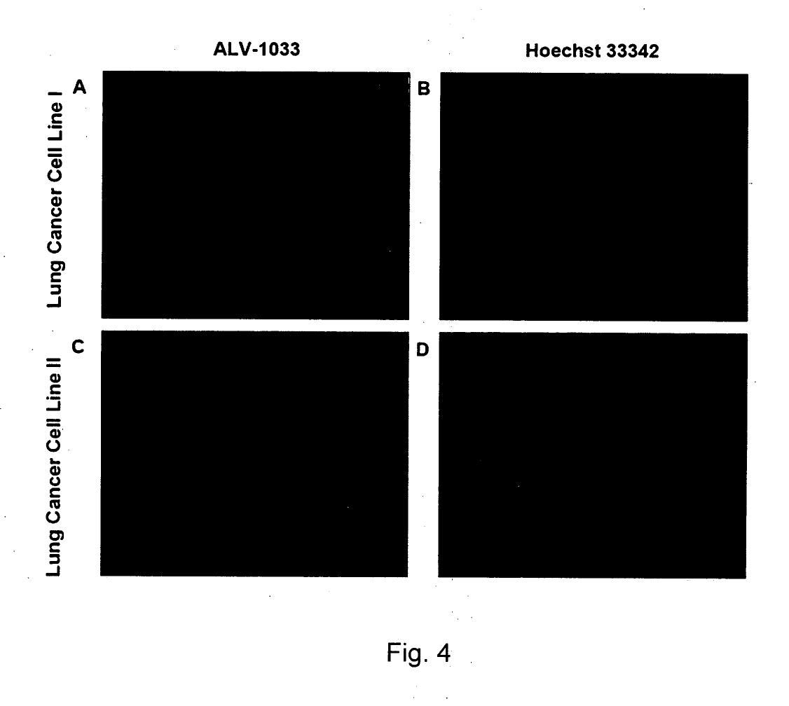

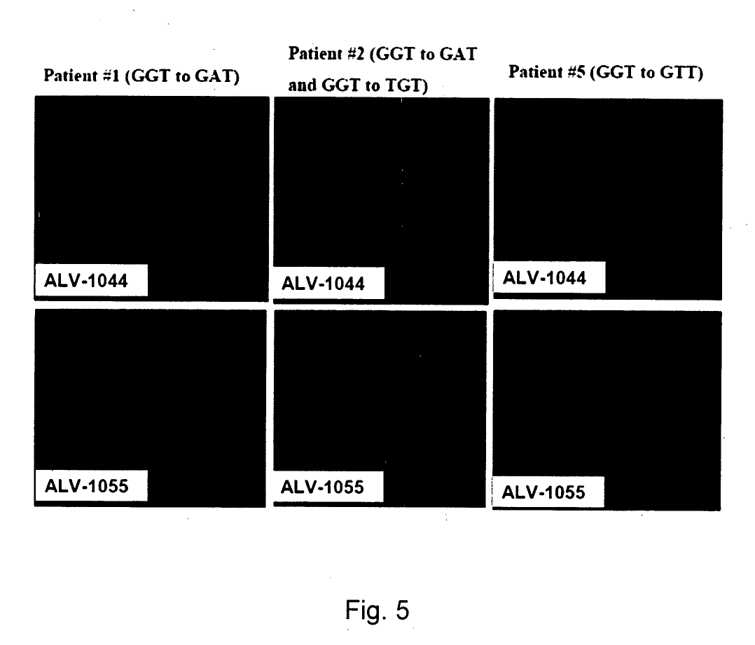

Detection of EGFR Mutations in Human Lung Cancer Cell Lines: The first products for cancer pharmacogenomics were designed to detect point mutations and/or deletions of EGFR in lung cancer. Specific mutation(s) of the targeted marker is known to correlate with the clinical response of patients undergoing EGFR-targeted therapeutic treatment. Results from preclinical studies, as shown in FIG. 4, indicates that the products of the invention detect point mutations in lung cancer cell line I (panel A), compared with wild type cell line II which does not have the mutations. The products of the invention can also detect specific deletions in EGFR marker gene. As shown in FIG. 5, the product detects deletion in a lung cancer cell line III (panel A), compared with the wild type cell line II which does not have the deletion in the targeted region of interest.

### Example 16

Detection of EGFR Mutations in Lung Cancer Patients: Feasibility studies using the products of the invention to detect EGFR mutations in cancer cells present in pleural fluids collected from NSCLC patients may be used to evaluate potentials of the products' cancer detection in clinical application for pharmacogenomics of EGFR targeted therapeutics. Representative data in FIG. 6 shows that the cancer product detected a deletion in EGFR tyrosin kinase domain in pleural fluid cancer cells collected from a NSCLC patient (red color, panel A). The patient was negative of EGFR point mutation as shown in panel B. The blue fluorescence is staining of nuclei of pleural fluid cells.

In summary, the detection molecules of the present invention for cancer pharmacogenomics are (1) able to simultaneously detect mutations as well as expression of specific; (2) therapeutic targets or markers from biological specimens; (3) designed for cancer pharmacogenomics and early cancer detection with specific marker expression; and (4) In possession of proof-of-concept demonstration in preclinical studies using cancer cell lines. The sample may be used include pleural fluid of SMCLC lung cancer patients.

### Example 17

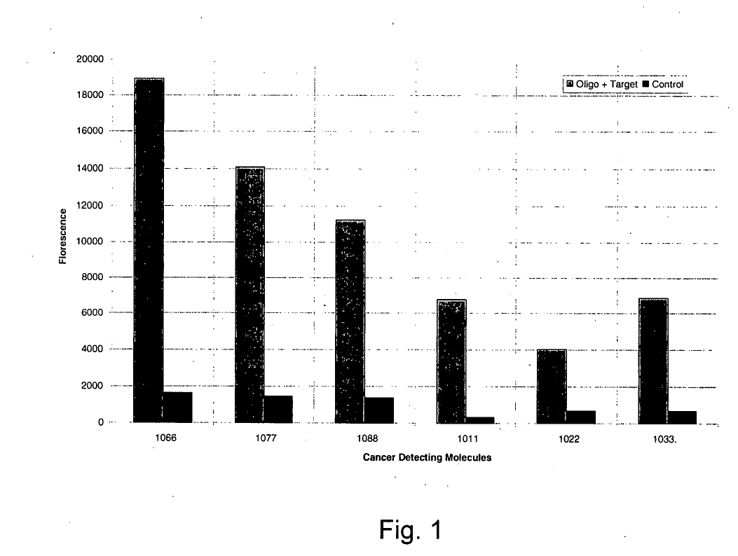

Cancer Detection: One aspect of the invention is related to developing molecules that are specific for detection of cancer markers and pharmacogenomic targets. A series of cancer detecting molecules were designed for the detection of cancer marker expression and of targets of cancer pharmacogenomics. As shown in FIG. 1, ALV-1011 and ALV-1022 were designed for the lung cancer pharmacogenomics. ALV-1033 was specific for the expression of a universal cancer marker. ALV-1066 and ALV-1077 were designed for detection of point mutations of a specific marker of pancreatic cancer.

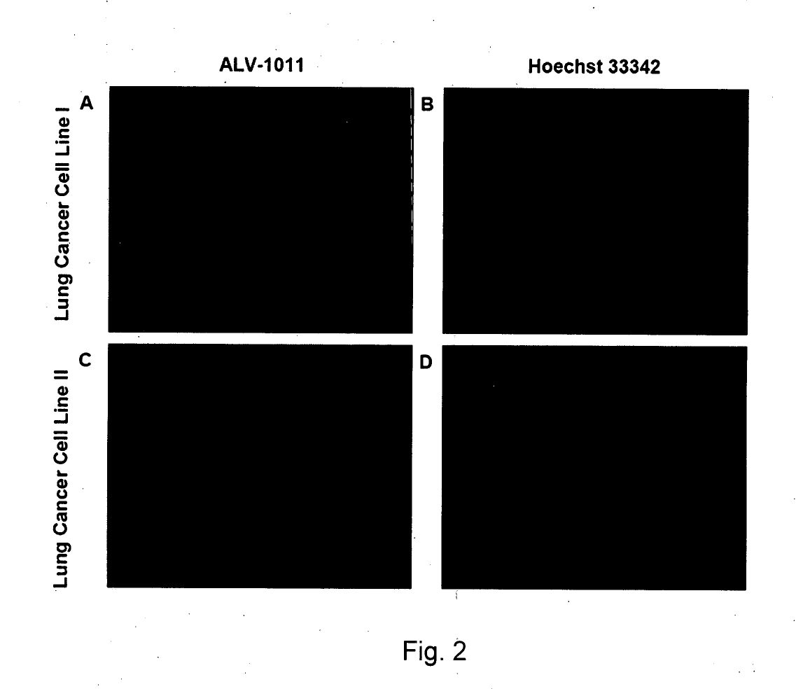

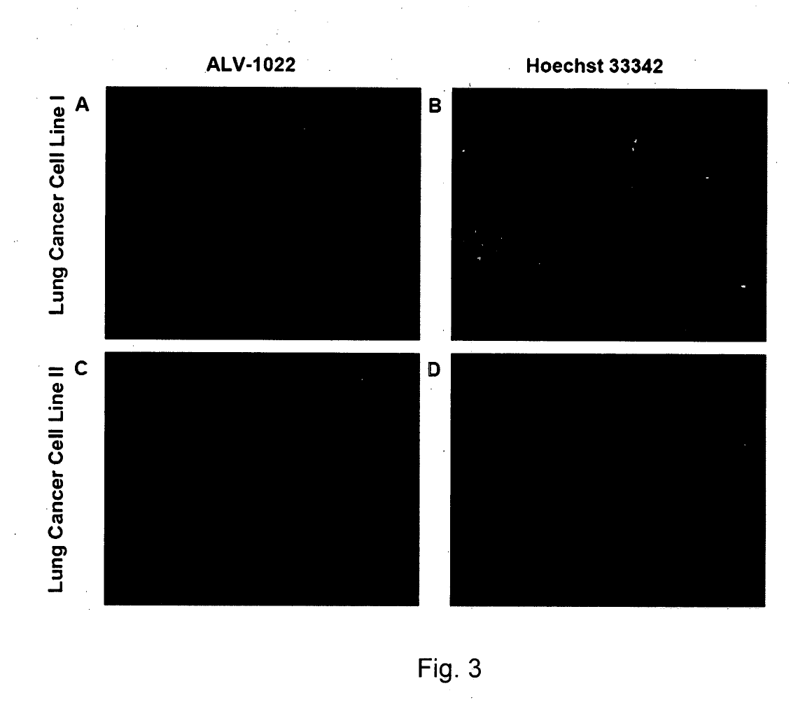

ALV-1011 and ALV-1022 were designed to detect a single mutation and/or deletion of a targeted lung cancer marker. Specific mutation(s) of the targeted marker is known to correlate with the clinical response of patients undergoing therapeutic treatment. Results from preclinical studies, as shown in FIGS. 2 and 3, indicated that point mutations in the lung cancer cell line I could be detected with integrity by ALV-1011 and ALV-1022 (panel A), respectively, compared with the cell line II which does not have the mutation.

ALV-1033 was designed to detect the expression of a “universal” cancer marker in the early stage of oncogenesis. Expression of the “universal” cancer marker was found in more than 80% of almost all kind of tumors and its level of expression is correlated with the prognosis of patient's disease progression. Expression of the “universal” cancer marker was usually undetectable in normal tissues. As shown in FIG. 4, ALV-1033 detected expression of the specific marker in the lung cancer cell line I (high) and II (low).

ALV-1033 is particularly useful in the diagnosis of breast cancer and lung cancer. Application of ALV-1033 may be used for diagnosis of other cancer indications, including colon and prostate cancers.

ALV-1044 and ALV-1055 were designed for early detection of pancreatic cancer. Mutation(s) of the marker occurs very early in the development of pancreatic cancer. Point mutations of the marker were found in >90% of pancreatic carcinomas. Most of these mutations were concentrated at a specific locus. Results in FIG. 5 demonstrated that ALV-1044 and ALV-1055 detected their specific targeted mutation in a specific cancer marker in biopsies from three individual pancreatic cancer patients.

Detection of the expression of multiple tumor marker genes simultaneously provides a specific and sensitive method for identification and classification of cancer cells in clinical samples such as tissue sections, aspirates from fine needle biopsy, blood and exfoliated cells in body fluids. According to one embodiment of the present invention, a portfolio of genes their expression associated with tumors of metastasis was identified by the products and methods of the invention.

The present invention, among other things, discloses methods that utilize molecular beacon imaging for detecting and/or identifying the presence of, point mutations of, and/or alterations in gene expression of, various cancer and virus markers in cells and tissues of a living subject, and applications of same. The molecular beacons, according to the present invention, are designed such that when one of the molecular beacons targets a disease-specific marker sequence in one or more cells, the fluorophore of the molecular beacon fluoresces, thereby generating a corresponding fluorescent signal. The fluorescent signal is detectable without a need of signal amplification.

According to the present invention, using the MBs to detect infections and expression or mutations of disease markers for diagnostics and pharmacogenomics by directly adding the MBs (reagents) to the specimens (the sample of cells), there is no need to perform signal amplification. It has been shown that USMD technology based assay is a rapid, specific, sensitive, easy-to-use and cost effective detection to a specific molecular target. Comparison of the invention with the diagnostic products currently available on the market, e.g. RT-PCR and immuno based assays, as outlined in Table 8, indicates the superiority of the invention.

TABLE 8
Comparison of ALVitae Products with RT-PCR and Immuno Assays

| | | | ALVitae |
|---|---|---|---|
| Technology | RT-PCR | Immuno Assays | USMD |
| Molecular Target | DNA and/or RNA | Protein | RNA |
| Speed | Greater 6 hours to | 30 minutes to | 30 minutes |
| | days | hours | |
| Specificity | Very Specific | Specific | Very Specific |
| Sensitivity | Need | Better with | No Need of |
| | Amplification | Inclusion of 2nd | Amplification |
| | | Antibody | |
| Easy to Use | Multiple Steps | One Step to | One Step |
| | | Multiple Steps | |
| Response to Drug | Very Quick | Very Slow | Very Quick |
| Resistant | | | |
| Mutation | | | |
| Cost per Test | High | Moderate | Low |

Among other things, the present invention has clinical and economic benefits that are summarized as follows:
Rapid One-Step Assay That Is Sensitive, Specific, Simple To Use And Cost Effective: USMD based detection of flu virus infection and cancer is a rapid and simple one-step assay. The whole process may take only 30 minutes or less to complete, compared with the current standard RT-PCR assay that takes longer that 6 hours for flu assays and days for EGFR detection in lung cancer.
Easy Handling of Test Results: Without the requirement of expensive equipments, the results generated from USMD based assays are measured with instruments commonly used in the clinical and research laboratory. In addition to fluorescence microscopes, the results may also be measured with a Fluorescence Activated Cell Sorter (FACS), a machine routinely utilized to monitor white blood cell counts in HIV infected patients, and fluorescence plate readers, a standard machine for immuno fluorescent assays.
Multiple Products Developed for Infection Detection of Various Flu Virus Strains: as disclosed above, the present invention has great advantages in detection of flu A and flu B subtypes as well as flu A(H5) and flu A(N1) strains. With combination of ALV-FluA, ALV-FluAH5 and ALV-FluAN1, the contagious avian flu recently outbreaks in Southeastern Asia can be detected. The USMD platform technology is applicable to other subtype and strain specific flu viruses.
Quick Response to Outbreak of Drug Resistant Mutants: the present invention, whether for cancer or flu infection, is utilized to detect mutations including deletions and point mutations. Should the outbreak of drug resistant mutants emerge, e.g. Tamiflu resistant strain of avian flu virus occurs or drug resistant cancer, the turn around time it takes to design and produce USMD based products is in the range of 2-3 weeks, once the mutated sequences are identified. The quick turn around time for the readiness of a new product is incomparable to that of antibody based assay development.
Applicable for Early Diagnostic Detection and Pharmacogenomics: the present invention is utilized to detect not only the expression of marker genes that are associated with disease progression such as in cancer and infectious diseases, but also deletions or point mutations that are correlated to the pharmacogenomics of targeted therapeutics. Both the early diagnostics and pharmacogenomics may benefit patients with early start of effective therapeutic treatment.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

### LIST OF REFERENCES

[1] Baselga J, Norton L. Focus on breast cancer. Cancer Cell 2002; 1(4):319-322.
[2] Belshe R B. The Origins of Pandemic Influenza—Lessons from the 1918 Virus. N Engl J Med 2005; 353(21):2209-2211.
[3] Giesendorf B A J et al. Molecular beacons: a new approach for semi-automated mutation analysis. Clin Chem 1998; 44:482-486.
[4] Hall I P. Pharmacogenetics, pharmacogenomics and airway disease. Respiratory Research 2002; 3:10.
[5] Hanahan D, Weinberg R A. The Hallmarks of Cancer. Cell 2000; 100(1);57-70.
[6] Leone G, van Schijndel H, van Gemen B, Kramer F R, Schoen C D. Molecular beacon probes combined with amplification by NASBA enable homogeneous, real-time detection of RNA. Nucleic Acids Res 1998; 26:2150-2155.
[7] Marras S A E, Kramer F R, Tyagi S. Multiplex detection of single-nucleotide variations using molecular beacons. Genet Anal 1999; 14:151-156.
[8] Kostrikis L G, Tyagi S, Mhlanga M M, Ho D D, Kramer F R. Spectral genotyping of human alleles. Science 1998; 279:1228-1229.
[9] Kostrikis L G et al. A chemokine receptor CCR2 allele delays HIV-1 disease progression and is associated with a CCR5 promoter mutation. Nat Med 1998;

4:350-353.
[10] Matsuo, T. In situ visualization of messenger RNA for basic fibroblast growth factor in living cells. Biochim Biophys Acta 1998; 1379:178-184.
[11] Minamoto T, Mai M, Ronai Z. K-ras mutation: early detection in molecular diagnosis and risk assessment of colorectal, pancreas, and lung cancers—a review. Cancer Detect Prev 2000; 24(1):1-12.
[12] Piatek AS et al. Molecular beacon sequence analysis for detecting drug resistance in Mycobacterium tuberculosis. Nat Biotechnol 1998; 16:359-363.
[13] Sokol D L, Zhang X, Lu P, Gewirtz A M. Real time detection of DNA-RNA hybridization in living cells. Proc Natl Acad Sci USA 1998; 95:11538-11543.
[14] Steemers F J, Ferguson J A, Walt D R. Screening unlabeled DNA targets with randomly ordered fiber-optic gene arrays. Nat Biotechnol 2000; 18:91-94.
[15] Tyagi S, Kramer F R. Molecular beacons: probes that fluoresce upon hybridization. Nat. Biotechnol 196; 14;303-308.
[16] Tyagi S, Bratu D P, Kramer F R. Multicolor molecular beacons for allele discrimination. Nat. Biotechnol 1998; 16:49-53.
[17] Vet JAM et al. Multiplex detection of four pathogenic retroviruses using molecular beacons. Proc Natl Acad Sci USA 1999; 96:6394-6399.
[18] Xiang-Hong Peng, Ze-Hong Cao, Jin-Tang Xia, Grant W. Carlson, Melinda M. Lewis, William C. Wood, and Lily Yang. Cabcer Res 2005; 65: (5), 1909-1917.

What is claimed is:

1. A method for detecting an infection and/or expression or a mutation of a disease marker for diagnostics and pharmacogenomics in a living subject comprising the steps of:
a) obtaining a specimen from the living subject, wherein the specimen contains one or more cells;
b) fixing specimen with an organic solvent;
c) adding a molecular beacon to the specimen; and
d) observing a result from adding the molecular beacon for detection of an infection and/or expression or a mutation of a disease marker,
wherein the molecular beacon is capable of hybridizing with a disease-related RNA or DNA of the disease marker in the one or more cells, thereby emitting a signal detectable without a need for signal amplification.

2. The method of claim 1, further comprising the step of staining at least one nuclei of one or more cells with a stain prior to the observing step.

3. The method of claim 1, wherein performing the steps of adding the molecular beacon and observing the result takes no more than 2 hours.

4. The method of claim 1, wherein the staining result is detectable with an instrument including one of a microscope, FACS scan, ELISA plate reader, Scanner, and any combinations thereof.

5. The method of claim 1, wherein the molecular beacon detects an infectious disease cell.

6. The method of claim 5, wherein the infectious disease comprises a flu virus disease.

7. The method of claim 6, wherein the flu virus includes a fluA virus.

8. The method of claim 7, wherein the fluA virus includes one of 16H and 9N strains, and any combinations thereof.

9. The method of claim 6, wherein the flu virus is selected from the group consisting of fluA, fluAH5, fluAN1, fluB, and any combinations thereof.

10. The method of claim 1, wherein the molecular beacon detects a cancer cell.

11. The method of claim 10, wherein the cancer is selected from the group consisting of lung cancer, liver cancer, stomach cancer, prostate cancer, breast cancer, pancreatic cancer, skin cancer, bone cancer, womb cancer, cervical cancer, brain cancer, colon cancer, throat cancer and any cancer occurred in an animal.

12. The method of claim 1, wherein the mutation is a point mutation and/or deletion of the disease marker.

13. The method of claim 1, wherein the disease marker is a biological target of a targeted therapeutics.

14. The method of claim 13, wherein the biological target is EGFR gene and/or a transcription product thereof.

15. The method of claim 14, wherein the EGFR gene contains a deletion mutation in EGFR tyrosine kinase domain.

16. The method of claim 1, wherein the molecular beacon comprises a single stranded hairpin shaped structured oligonucleotide probe containing a nucleotide sequence selected from the group consisting of SEQ ID NOs: 1-11, and any combinations thereof.

17. A method for detecting a cancer cell from a living subject comprising the steps of:
a) obtaining from the living subject a specimen containing one or more cells;
b) fixing the specimen with an organic solvent;
c) adding a molecular beacon into the specimen; and
d) observing a result from adding the molecular beacon for detection of the cancer cell in the specimen,
wherein the molecular beacon is capable of hybridizing with a cancer cell marker-related RNA or DNA in one or more cells in the specimen, thereby emitting a signal detectable without a need for signal amplification.

18. The method of claim 17, further comprising the step of staining a nuclei of one or more cells in the specimen with a stain prior to the observing step.

19. The method of claim 17, wherein the organic solvent is one of acetone, alcohol, methanol, formalin, paraformaldehyde, butanol, and any combinations thereof.

20. The method of claim 17, wherein the organic solvent fixed specimen is subject to a Triton treatment prior to the addition of the molecular beacon.

21. The method of claim 17, wherein the molecular beacon comprises a single stranded hairpin shaped structured oligonucleotide probe containing a nucleotide sequence selected from the group consisting of SEQ ID NOs: 1-7, and any combinations thereof.

22. The method of claim 17, wherein the cancer cell is selected from the group consisting of lung cancer, liver cancer, stomach cancer, prostate cancer, breast cancer, pancreatic cancer, skin cancer, bone cancer, womb cancer, cervical cancer, brain cancer, colon cancer, throat cancer and any cancer occurred in an animal.

23. The method of claim 17, wherein the cancer cell exhibits at least one point mutation and/or deletion in a specific marker of the cancer cell.

24. The method of claim 17, wherein the specimen is one of a tissue section, an aspirate from biopsy, blood, and an exfoliated cell in a body fluid.

25. A method for detecting a flu virus-infected cell from a living subject comprising the steps of:
a) obtaining from the living subject a specimen, wherein the specimen contains one or more cells;
b) fixing specimen with an organic solvent;
c) adding a molecular beacon into the specimen;
d) observing the result for detection of the flu virus-infected cell in the specimen.
wherein the molecular beacon is capable of hybridizing with a flu virus marker-related RNA or DNA in at least one cell, thereby emitting a signal detectable without a need for signal amplification.

26. The method of claim 25, further comprising the step of staining at least one nuclei of one or more cells with a stain prior to the observing step.

27. The method of claim 25, wherein the organic solvent is one of acetone, alcohol, methanol, formalin, paraformaldehyde, butanol, and any combinations thereof.

28. The method of claim 25, wherein the organic solvent fixed specimen is subject to a Triton treatment prior to addition of the molecular beacon.

29. The method of claim 25, wherein the molecular beacon comprises a single stranded hairpin shaped structured oligonucleotide probe containing a nucleotide sequence selected from the group consisting of SEQ ID NOs: 8-11, and any combinations thereof.

30. The method of claim 25, wherein the flu virus comprises an avian flu virus.

31. The method of claim 25, wherein the flu virus is a fluA or fluB virus.

32. The method of claim 31, wherein the fluA virus includes one of 16H and 9N strains, and any combinations thereof.

33. The method of claim 25, wherein one or more than one probe is added into the specimen simultaneously.

34. A method for detecting an infection and/or expression or a mutation of a disease marker for diagnostics and pharmacogenomics in a living subject comprising the steps of:
a) obtaining a specimen from the living subject, wherein the specimen contains one or more cells;
b) fixing specimen with an organic solvent;
c) adding a molecular beacon to the specimen;
d) observing a result from adding the molecular beacon for detection of infections and/or expression or a mutation of a disease marker,
wherein the molecular beacon is capable of hybridizing with a disease-related RNA or DNA of the disease marker in a cell, thereby emitting a signal detectable without a need for signal amplification.

35. The method of claim 34 further comprising the step of staining a nuclei of one or more cells in the specimen with a stain prior to the observing step.

36. The method of claim 34, wherein performing adding the molecular beacon and observing the result take no more than 2 hours.

37. The method of claim 34, wherein the organic solvent is one of acetone, alcohol, methanol, formalin, paraformaldehyde, butanol, and any combinations thereof.

38. A molecular beacon comprising a single stranded hairpin shaped structured oligonucleotide probe containing a nucleotide sequence capable of hybridizing with a disease-related RNA and/or DNA of a disease marker in a disease cell, thereby emitting a signal detectable without a need for signal amplification.

39. The molecular beacon of claim 38, wherein the oligonucleotide probe contains a nucleotide sequence selected from the group consisting of SEQ ID NOs:1-11.

40. The molecular beacon of claim 38, wherein the oligonucleotide probe has a nucleotide sequence capable of hybridizing with RNA and/or DNA encoding EGFR gene tyrosine kinase domain in a cancer cell.

41. The molecular beacon of claim 38, wherein the oligonucleotide probe comprises a fluorofore at 5′ and a quencher at 3′, or a fluorofore at 3′ and a quencher at 5′.

42. The molecular beacon of claim 38, wherein the disease cell is one of a cancer cell or an infectious disease cell.

43. The molecular beacon of claim 38, wherein the disease cell is infected by a flu virus.

44. The molecular beacon of claim 38, wherein the flu virus is a fluA or fluB virus.

45. The molecular beacon of claim 44, wherein the fluA virus includes one of 16H and 9N strains, and any combinations thereof.

46. The molecular beacon of claim 38, wherein the disease cell is a cancer cell.

47. A. diagnostic kit for detecting an infection and/or expression or a mutation of a disease marker for diagnostics and pharmacogenomics in a living subject comprising:
a) a molecular beacon of claim 38; and
b) an instruction sheet.

48. The diagnostic kit of claim 47, wherein the oligonucleotide probe contains a nucleotide sequence capable of hybridizing with RNA and/or DNA encoding EGFR gene tyrosine kinase domain in a cancer cell.

49. The diagnostic kit of claim 47, wherein the oligonucleotide probe contains a nucleotide sequence selected from the group consisting of SEQ ID NOs: 1-11.

50. The diagnostic kit of claim 47, wherein the kit comprises more than one oligonucleotide probe containing a nucleotide sequence selected from the group consisting of SEQ ID NOs: 1-7.

51. The diagnostic kit of claim 47, wherein the kit comprises more than oligonucleotide probe containing a nucleotide sequence selected from the group consisting of SEQ ID NOs: 8-11.

52. The diagnostic kit of claim 47, wherein the performance of diagnosis from adding the molecular beacon into a specimen to observing a result therefrom takes no more than 2 hours.

53. The molecular beacon of claim 38, wherein the oligonucleotide probe contains a nucleotide sequence capable of hybridizing with RNA and/or DNA encoding a universal cancer marker.

54. The method of claim 1, wherein the molecular beacon is capable of hybridizing with a transcription product of EGFR.

55. The method of claim 1, wherein the molecular beacon is capable of detecting a drug-resistant cancer and/or a drug-resistant pathogen.
